(19) United States
(12) Patent Application Publication
(10) Pub. No.: US 2018/0126053 A1
(43) Pub. Date: May 10, 2018

(54) WRISTWATCH FOR MONITORING OPERATION OF AN IMPLANTED VENTRICULAR ASSIST DEVICE

(71) Applicant: Leviticus Cardio Ltd., Petach Tikva (IL)

(72) Inventor: Michael Zilbershlag, Givat Shmuel (IL)

(21) Appl. No.: 15/861,987

(22) Filed: Jan. 4, 2018

Related U.S. Application Data
(63) Continuation-in-part of application No. 15/713,066, filed on Sep. 22, 2017.
Application No. 15/713,066 is a continuation-in-part of application No. 14/535,528, filed on Nov. 7, 2014, now Pat. No. 9,793,579.
Application No. 14/535,528 is a continuation-in-part of application No. 15/481,199, filed on Apr. 6, 2017.
Application No. 15/481,199 is a continuation-in-part of application No. 14/041,698, filed on Sep. 30, 2013, now Pat. No. 9,642,958.
Application No. 14/041,698 is a continuation-in-part of application No. 13/588,524, filed on Aug. 17, 2012, now Pat. No. 9,343,224.
(60) Provisional application No. 61/901,751, filed on Nov. 8, 2013.
(60) Provisional application No. 61/708,333, filed on Oct. 1, 2012.
(60) Provisional application No. 61/540,140, filed on Sep. 28, 2011.
(60) Provisional application No. 61/525,272, filed on Aug. 19, 2011.

(51) Int. Cl. A61M 1/10; A61M 1/12

(52) U.S. Cl. CPC A61M 1/1086; A61M 2205/8243; A61M 1/127; A61M 2205/04; A61M 2205/17; A61M 2205/18; A61M 2205/3523; A61M 2205/3584; A61M 2205/505; A61M 2205/52; A61M 2205/582; A61M 2205/581; A61M 2205/583; A61M 2205/8206; A61M 2205/8287; A61M 1/122

(57) ABSTRACT

A wristwatch wirelessly connected to an implanted medical device such as a VAD is a component of a coplanar energy transfer system. The wristwatch monitors the operation and performance of the VAD or its battery and provides alerts to potentially dangerous situations. The watch can receive signals related to, for example, the current status of the implant (e.g., operating metrics, energy demand, etc.) and current status of the internal battery (e.g., remaining useful life of battery, battery faults, etc.). The wristwatch serves as a redundant external controller of the implanted VAD. The user can interface with the wristwatch to send commands to the VAD and control its performance, power, or charging characteristics with the push of a button. The wristwatch also includes alarm features, which indicate to the user when a fault has occurred or whether there is some situation that requires medical attention.

### CROSS-REFERENCE TO RELATED APPLICATIONS

This application is a continuation-in-part of U.S. patent application Ser. No. 15/713,066, filed Sep. 22, 2017, which is a continuation-in-part of U.S. patent application Ser. No. 14/535,528, filed Nov. 7, 2014 (which issued as U.S. Pat. No. 9,793,579 on Oct. 17, 2017), which claims the benefit of and priority to U.S. Provisional Application Ser. No. 61/901,751, filed Nov. 8, 2013.

This application is also a continuation-in-part of U.S. patent application Ser. No. 15/481,199, filed Apr. 6, 2017, which is a continuation-in-part of U.S. patent application Ser. No. 14/041,698, filed Sep. 30, 2013 (which issued as U.S. Pat. No. 9,642,958 on May 9, 2017), which claims the benefit of and priority to U.S. Provisional Application Ser. No. 61/708,333, filed Oct. 1, 2012. U.S. patent application Ser. No. 14/041,698 is also a continuation-in-part of U.S. patent application Ser. No. 13/588,524, filed Aug. 17, 2012 (which issued as U.S. Pat. No. 9,343,224 on May 17, 2016), which claims the benefit of and priority to U.S. Provisional Application Ser. No. 61/540,140, filed Sep. 28, 2011, and 61/525,272, filed Aug. 19, 2011.

The entire contents of each of the above-referenced applications is incorporated herein by reference.

### FIELD

The present invention generally relates to wireless energy transfer into the body of a patient to wirelessly power a device implanted within the body and wireless monitoring and tracking of various operational parameters associated with the implanted device.

### BACKGROUND

Active implanted medical devices with chargeable batteries need constant monitoring. Ventricular assist devices (VADs) or full artificial hearts that operate using secondary re-chargeable batteries that receive power from an external transmitter can be monitored using an external device configured to communicate with the VAD and report various operating parameters of the VAD or an implanted battery. Devices for monitoring the operation of implanted devices are important for informing the user about the performance of the implant generally and alerting the user to potentially dangerous conditions in particular. A convenient device for monitoring and reporting the operating parameters of an implanted VAD is a wristwatch, which can be configured to display remaining battery life of the VAD, operating speed of the VAD, and other useful information.

### SUMMARY

The present disclosure relates to wristwatches that provide monitoring functions for implanted VADs. The disclosed wristwatches display performance characteristics of the VAD or its battery and provide alerts to potentially dangerous situations, while generally hiding or disguising the functionality so that the device appears to be a regular wristwatch. In other words, the wristwatch does not call attention to the fact that the user has an implanted device of some kind. Additionally, the wristwatch serves as a redundant external controller of the implanted VAD. The user can interface with the wristwatch to send commands to the VAD and control its performance, power, or charging characteristics with the push of a button. The wristwatch is also useful for its alarm features, which indicate to the user when a fault has occurred or whether there is some situation that requires medical attention. The wristwatch can alert the user to the situation and guide the user through remediation steps as applicable.

In particular the wristwatch can provide an alert that indicates a VAD malfunction (such as a suction event, a VAD partial failure, or VAD failure) or a battery malfunction. The implanted active controller can use the wristwatch as a first gentler alarm to alert the user to a problem, and can also employ a secondary more intense alarm such as vibration or electrical shock.

The disclosed wristwatches are applicable for use with left ventricular assist devices (LVADs), right ventricular assist devices (RVADs), biventricular assist devices (BVADs) and full artificial hearts alike. In the present disclosure, the term VAD encompasses all types of ventricular assist devices. When a particular type of VAD such as an LVAD is specified, it would be understood by a person of ordinary skill in the art how the same could apply to an RVAD, BVAD, or a full artificial heart.

Devices of the invention can take the form of any wristwatch known in the art, including smartwatches. In general, the device is portable and wearable, and it is configured to wirelessly communicate with at least the internal controller and receive data therefrom associated with the operational parameters of the implant and internal battery. Such operational parameters may include, for example, the current status of the implant (e.g., operating metrics, energy demand, etc.) and current status of the internal battery (e.g., remaining useful life of battery, battery faults, etc.).

As will be described in greater detail below, the wristwatch can be used in conjunction with a coplanar energy transfer system that includes an external transmission belt. A patient may not wish to wear the external assembly, as it may be cumbersome and uncomfortable to wear over extended periods of time (e.g., during the day). Accordingly, when the external assembly is not in use (e.g., patient takes off the transmission belt), the internal controller may rely on the internal battery for an energy supply. In such instances, rather than relying on the external controller as a means of providing informational data, the smart watch may be used to collect data from the internal controller and further output informational data to the patient or medical professional providing care to the patient. Accordingly, any crucial information related to operational parameters of the implant and/or internal battery can be communicated to the patient or medical professional without the need for the patient to wear, or otherwise carry, the external controller.

For example, the smart watch may be configured to provide alerts to a patient so as to warn the patient (or medical professional) of any critical information related to the implant or internal battery. For example, based on data received from the internal controller, the smart watch may provide at least one of a visual, audible, and haptic alert indicating a potential failure in the implant or battery. For example, data received from the internal controller may relate to the internal battery having had a fault or potentially having a fault in the future, as well as the amount of useful battery life remaining, thereby indicating a recharge is necessary. The alert may include information about the condition of the battery, such as that it has exceeded a threshold of probability to stop working or to explode. Such warnings can serve to alert the patient to secure some backup power source, or it can alert the patient that surgery is required to replace the defective battery.

In certain aspects, the invention involves a system for monitoring an implantable ventricular assist device (VAD) in a patient. The system includes an implantable assembly comprising a controller and a battery. The controller is configured to provide power from the battery to the implantable VAD and collect data associated with at least one of the implantable VAD and the battery. The system also includes a wristwatch, such as a smart watch, that has a wireless receiver configured to wirelessly receive the data from the controller and a data output component, such as a visual display, an audio source, and/or a haptic feedback source, configured to present information to a user based on the received data. The information presented by the data output component can be an alert indicating an event that is life-threatening to the patient. The alert can be visual, audible, haptic, or a combination thereof. In a related aspect, the invention is the system described above, but for monitoring an artificial heart.

In embodiments, the system also includes an external transmission inductive coil and an external controller, and an internal receiver inductive coil coupled to the VAD and configured to receive wirelessly transmitted energy from the external transmission inductive coil. In other embodiments, the implantable assembly includes a receiver inductive coil coupled to the controller and configured to wirelessly receive inductively-transferred electromagnetic power from a non-implanted power source and provide power to the implanted implantable VAD via the controller.

The information based on the received data can include information associated with operational or performance characteristics of the implanted medical device or the battery. Those operational or performance characteristics may be operating metrics, energy demand, remaining useful life, fault potential, and a combination of at least two thereof.

The controller and wristwatch are configured to wirelessly transmit data via a wireless transmission protocol selected from the group consisting of Bluetooth communication, infrared communication, near field communication (NFC), radio-frequency identification (RFID) communication, WiFi, and cellular network communication. The controller and the wristwatch may be configured to wirelessly transmit data via the frequency band of the Medical Implant Communication Service (MICS), or Medical Device Radiocommunications Service (MedRadio). In certain embodiments, the wristwatch serves as a protocol bridge between the implant and off-the-shelf computing device using MICS or MedRadio to communicate with the implant controller and Bluetooth or WiFi to communicate to the off-the-shelf computing device.

In other aspects, the invention includes a wristwatch for monitoring operation of an implanted VAD. The wristwatch includes a wireless receiver configured to pair with a transmitter implanted within a patient and receive from the transmitter data related to operating parameters of the implanted VAD or an associated implanted battery. The wristwatch also includes a data output module configured to alert a user when the operating parameters are indicative of a life-threatening event.

In embodiments, the operating parameters include operating metrics, energy demand, remaining useful life, fault potential, battery capacity, battery capacitance, battery voltage, battery power, or any combination thereof. The alert includes a visual, audible, haptic alert, or a combination thereof. The alert may include an amount of time remaining before failure of the implanted battery.

In certain embodiments, the transmitter is configured to wirelessly transmit data to the non-implanted wireless receiver via a wireless transmission protocol selected from the group consisting of Bluetooth communication, infrared communication, near field communication (NFC), radio-frequency identification (RFID) communication, wifi, and cellular network communication. The transmitter may be configured to wirelessly transmit data to the non-implanted wireless receiver via the frequency band of the Medical Implant Communication Service (MICS) or Medical Device Radiocommunications Service (MedRadio). The wristwatch may serve as a protocol bridge between the implant and off-the-shelf computing device using MICS or MedRadio to communicate with the implant controller and Bluetooth or WiFi to communicate to the off-the-shelf computing device.

In a related aspect, the invention involves a system for monitoring operation of an implanted left ventricular assist device (LVAD) and an implanted battery for conveying blood through a human heart of a patient. The system includes a wristwatch containing a wireless receiver configured to pair with a transmitter implanted within the patient, and receive from at least one implanted processor associated with the transmitter, indications of operating parameters of the LVAD including an indication of an amount of time remaining until reconnection to an external power source is required, and warning signals relating to at least one dangerous state of the LVAD. The system also includes an alarm in the wristwatch for alerting the patient to a life-threatening event during operation of the LVAD.

In embodiments, the system includes a display on the wristwatch for providing feedback on operation of the LVAD. The feedback may include an indicator of an amount of time remaining until the patient is required to reconnect to an external power source. The indicator of the amount of time may be a display of remaining capacity, capacitance, or voltage of the implanted battery. The wristwatch may also include at least one processor for causing to appear on the display instructions to the patient for taking corrective action to mitigate the event. The event may include a high power event, low implanted battery power, a cessation of operation of the LVAD, or a failure that requires use of redundancy mechanism. The event may include a disconnection of a connector, a failure of an implanted battery, or a failure of an engine in the LVAD.

In some embodiments, the wristwatch is configured to establish an authenticated secure connection and/or an encrypted connection with the implant controller.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1A is a smart watch for use with the invention.

FIG. 1B is a schematic overview of the wireless coplanar energy transfer (CET) system according to certain embodiments.

FIG. 2 illustrates a wireless CET system consistent with the present disclosure.

FIG. 3 is a block diagram illustrating the wireless CET system of FIG. 2.

FIG. 4 depicts an embodiment of the receiver inductive coil consistent with the present disclosure.

FIG. 5 depicts an embodiment of the internal controller including an internal battery consistent with the present disclosure.

FIG. 6 depicts an embodiment of the external assembly of the system, including the transmission belt (having the transmitter inductive coil), the external controller and external battery consistent with the present disclosure.

FIG. 7 is a block diagram illustrating the external controller in greater detail.

FIG. 8 is a block diagram illustrating the internal controller in greater detail.

FIG. 9 is a block diagram illustrating the user smart watch in greater detail.

FIG. 10 is a schematic overview of the process of encrypted communication of data between the internal controller and one of the external controller, the user smart watch, and the user tablet computing device.

FIG. 11 is a block diagram illustrating the communication of informational data from the internal controller to the user smart watch and the output of alerts to a user based on the informational data.

FIG. 12 shows a diagram of a battery system consistent with one embodiment of the present disclosure.

FIG. 13 shows a circuit diagram of a battery system for providing cell redundancy and thermal run away prediction consistent with the present disclosure.

FIG. 14 depicts one embodiment of a transmission belt surrounding an implantable receiver coil.

FIG. 15 illustrates an external belt (such as the belt in FIG. 1) worn by an individual.

FIG. 16 shows an implantable circuit with a DC-to-DC converter coupled to the implantable receiver resonance structure

FIG. 17 is a graph showing a relationship between output power and load resistance.

FIG. 18 shows an alternative circuit coupled to the implantable receiver coil, wherein the circuit has the same resonance structure but uses the inductiveness of the implant and controller as a DC to DC.

FIG. 19 is a circuit that can be used to lock the external transmitter resonance frequency to the implanted receiver resonance frequency.

FIG. 20 is another circuit that can be used to lock the external transmitter resonance frequency to the implanted receiver resonance frequency.

FIG. 21 shows ring coil implanted in the bottom of the pericardium sack.

FIG. 22 shows two ring coil implanted in the bottom of the pulmonary cage.

FIG. 23 shows stent base ring coil located in the descending aorta.

FIG. 24 shows a model circuit for calculating the efficiency of energy transmission.

FIG. 25 shows a schematic of a transmitter circuit.

FIG. 26 shows a schematic of a parallel-loaded receiver circuit.

FIG. 27 shows a schematic whereby a simple single-phase rectifying circuit is added to a receiver.

FIG. 28 is a flow chart of coarse and fine resonance frequency detection.

FIG. 29 is a flow chart of coarse and fine frequency base power control.

FIG. 30 shows a configurable capacitor for use with a circuit for locking the external transmitter resonance.

FIG. 31 exemplifies frequencies that can cause tissue heating.

FIG. 32 depicts dynamic band of frequency used to search for resonance or target frequency.

FIG. 33 illustrates the effect that the height of a transmitter coil has on robustness of power transfer if the proximity effects are ignored.

FIG. 34 illustrates the effect that the height of a transmitter coil has when the proximity effects are considered.

FIG. 35 depicts a preferred circuit for the half bridge Pulse generator.

FIG. 36 illustrates rectification with one diode (half wave) and with a diode bridge (full wave).

FIG. 37 illustrates the geometry of the transmitter coil and receiver coil and their relation to each other.

FIG. 38 depicts a transmitter coil having 16 wire turns arranged in two layers (e.g. 8 turns per layer).

FIG. 39 depicts a separated ring coil according to certain embodiments.

FIG. 40 depicts a separated ring coil disposed within a pleural cavity and pericardium.

### DETAILED DESCRIPTION

Wristwatches of the present invention are configured to communicate wirelessly with implanted VADs. The wristwatches receive information from implanted components indicative of performance parameters of the VAD or battery, and display that information on a screen for the user to observe. The wristwatch is also configured to accept inputs from the user, which allows the user to send commands to the implanted components to control their operation.

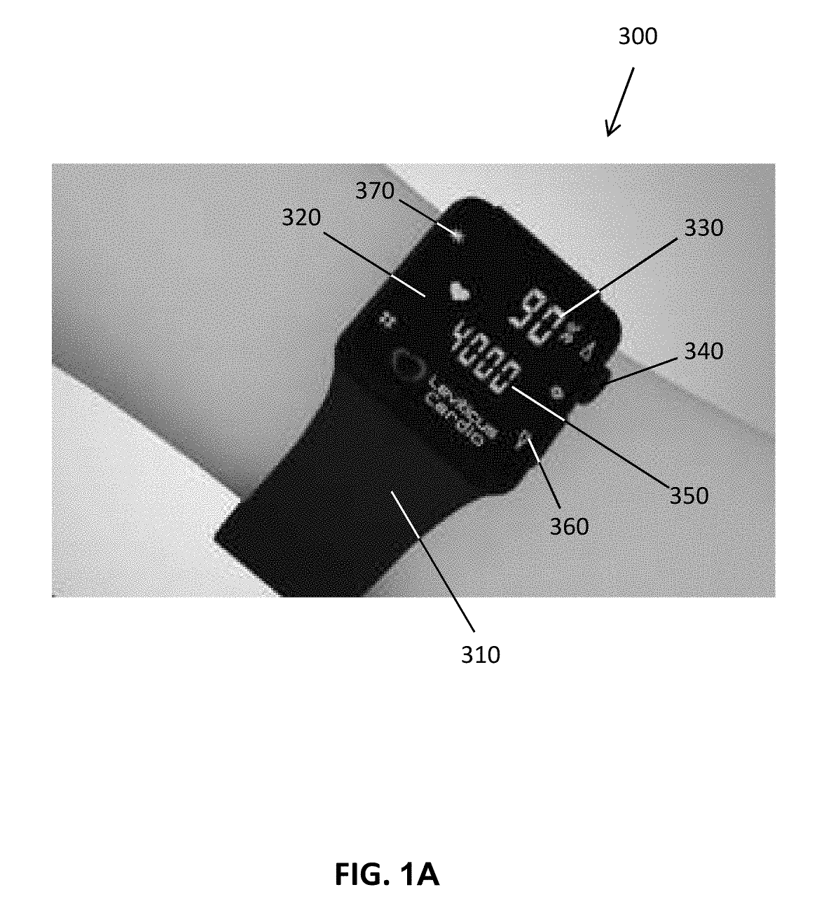

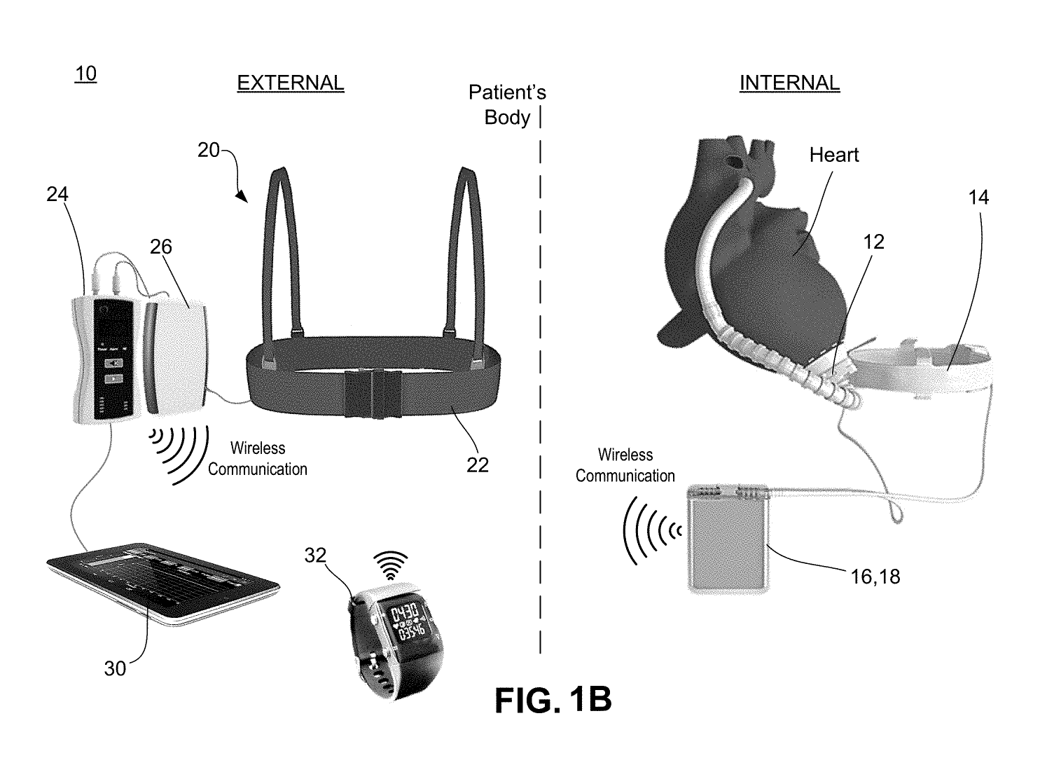

An exemplary wristwatch is shown in FIG. 1A. The wristwatch 300 is generally a smart watch configured to communicate with the internal transmitter. The wristwatch 300 is configured to wirelessly pair with a transmitter implanted in the patient. The transmitter is associated with one or more processors that are configured to send indications of the operating parameters of the VAD to the smart watch.

The wristwatch 300 may be configured to establish an authenticated secure connection or an encrypted connection with the implant controller. The secure or encrypted connection helps reduce the risk of the device being hacked or having its data compromised in some way. This feature also helps to ensure that the function of the watch is not interfered with inadvertently, such as by receiving signals from another device, such as a VAD implanted in another person nearby.

The wristwatch 300 is configured to receive regular indications of the operating parameters of the VAD, and also provide warnings to the user when a parameter has reached an unsafe level.

The operating parameters that the transmitter sends to the wristwatch 300 may include an amount of battery power remaining, which may be expressed as an amount of time remaining before reconnection to an external power source is required. The wristwatch 300 may display capacity, capacitance, or voltage of the implanted battery.

The disclosed wristwatches provide various benefits besides merely displaying performance characteristics of the VAD or its battery and providing alerts to potentially dangerous situations. The wristwatch serves as a redundant external controller of the implanted VAD. The user can interface with the wristwatch to send commands to the VAD and control its performance, power, or charging characteristics with the push of a button. The wristwatch is also useful for its alarm features, which indicate to the user when a fault has occurred or whether there is some situation that requires medical attention. The wristwatch can alert the user to the situation and guide the user through remediation steps as applicable. Additionally, the wristwatch may be designed to generally hide or disguise the functionality so that the device appears to be a regular wristwatch. In other words, the wristwatch does not call attention to the fact that the user has an implanted device of some kind.

The wristwatch 300 includes a watch band 310 connected to a watch face with a screen 320 that is configured to display various operating parameters of the VAD. For example, the screen 320 may display a field 330 showing the percent battery life remaining in the VAD. Optionally the wristwatch 300 can display the operating parameters of other components of the system, including the watch itself, by including another field (not shown) that displays the battery life remaining. The watch screen 320 may include other symbols that indicate the quality of the wireless connection with other apparatuses. The watch screen 320 further includes a field 350 that shows the speed at which the VAD is operating. In some embodiments, the wristwatch 300 may display several different parameters in the same field, and may be configured to scroll through the various parameters at regular intervals. The dial 340 may be used for various input functions, such as scrolling through the watch functions or provide other user inputs. The dial 340 may be a multi-functional button. The wristwatch 300 may also be configured to change which parameter is displayed in response to the user engaging the dial function or the button function of the dial 340. The screen 320 may be a touch screen configured to receive commands corresponding to tactile contact from the user. The screen 320 may include other hot keys 360 that accept various user inputs for interacting with the display. The screen 320 may also include other icons 370 for displaying various information.

The wristwatch 300 can also include a processor (not shown) for causing instructions to appear on the display screen 320, which alert the user to a recommended course of corrective action in the event of a failure or other potentially dangerous event. The watch may include internet capability or GPS capability, which would assist the device in, for example, alerting the user to the nearest hospital in the event of a life-threatening emergency.

In addition to monitoring the regular operating parameters, the processor is further configured to send warning signals to the external wristwatch 300 to indicate an event that requires the user's attention. The event may be any dangerous operating state of the VAD, such as failure of the implanted battery, a short in one of the battery cells, failure of an engine in the VAD, or a loss of power to the VAD. The event may be a high power event or a warning about low power in the implanted battery. The event may be the cessation of operation of the VAD. The event may be a failure that causes a redundancy mechanism.

When the event is detected, the processor is configured to send a signal indicating that an event has occurred (or is likely to occur). The watch may alert the user to the current operating condition of the battery, such as that a cell has failed or that a backup cell has begun operating.

The watch shows the status of the VAD, including its battery life and operating speed. Additionally, the watch is configured to initiate an alert or alarm, which may be auditory, haptic, kinesthetic, visual, and the like, or a combination of the above. Different types of alerts can be configured for different purposes. For example, as the remaining battery power declines, the alerts can become more intense (i.e., louder, stronger vibration, stronger electrical shock, etc.) to indicate the increasing level of severity of the danger.

In various embodiments, the watch may be configured with additional buttons, switches, hot keys, or the like, which can be used by the wearer to acknowledge the alert and turn the alert off. The watch may also be configured to provide a periodic reminder (in the form of a new alert, for example) if the cause for the original alert is not addressed after a predetermined period of time. For example, if the alert indicates that the VAD battery is low and the user acknowledges the alert by turning it off, the smart watch may provide another alert after a predetermined period of time if the battery has not been replaced or recharged. As long as the event remains, the watch continues to warn the user periodically.

In some embodiments, the alert may increase in intensity until it is acknowledged by the user. For example, if the alert is an auditory alert such as a beeping sound, the beep may start at a low volume and gradually increase. Alternatively, the beeping can start off slow and gradually become faster until the user acknowledges the alert.

Wristwatches of the invention can be part of a wireless coplanar energy system shown in FIG. 1B. Implanted medical devices such as VADs and their associated coplanar energy transfer systems will be described in further detail. It should be understood that the wristwatches described above are compatible for use with the systems described below.

As shown in FIG. 1B, a wristwatch 32 can be incorporated into a system for powering an implanted medical device 12. The device 12 is generally configured to communicate with an external controller 22. As a safety feature, the implant may send an alert indicating that the battery is low to the controller 22. However, this safety feature only works when the system is receiving power from an external source, and so what is needed is an added alert system when the external power source is absent.

To address this problem, and more generally to address the risk that an implanted battery pack may lose energy before a VAD patient is able to reconnect to an external power source, an external wristwatch 32, which may be a smart watch, is provided that provides feedback and warnings of life-threatening conditions such as a low battery or loss of power. The wristwatch 32 is configured to connect wirelessly with an internal transmitter in order to monitor the VAD function in real-time. The wristwatch 32 provides a display that can be observed by a user and additionally provides alerts to notify the user when certain operating conditions of the VAD reach unsafe levels or are determined to have an undue risk of reaching such levels. The smart watch can be a dedicated device for use with the VAD, or it can be another commercially available smart watch capable of installing and running a mobile app that is compatible with the disclosed VAD systems.

Wireless Coplanar Enemy Transfer Systems

Watches described above can be used with wireless coplanar energy transfer (CET) systems. The various embodiments of CET systems of the present invention include an internal assembly, generally including a medical implant, such as a ventricular assist device (VAD), to be implanted within the body of a patient and an external assembly configured to wirelessly deliver energy to the internal assembly and ultimately provide power to the implant. In particular, the internal assembly generally includes a medical implant, an internal controller and a receiver inductive coil coupled to the implant, and optionally an internal battery configured to store energy for the purpose of providing backup power to the implant. The external assembly generally includes a transmission belt (which includes a transmitter inductive coil), an external controller, and optionally an external battery.

The transmission belt is designed to be placed externally around a part of the patient's body such that the transmitter inductive coil is disposed in a coplanar manner with the receiver inductive coil to allow for wireless energy transfer from the transmitter inductive coil to the receiver inductive coil. During operation of the CET system, the external controller is configured to draw energy from the external battery and control transmission of such energy to the transmission belt, which, in turn, is configured to wirelessly transmit power through the patient's body to the internal receiver coil via the magnetic coupling. Upon receiving energy, the receiver inductive coil is configured to transmit such energy to the internal controller, which, in turn, is configured to drive, or control operation of, the implant, as well as provide energy to the internal battery (e.g., charge the internal battery).

Furthermore, the internal controller is configured to wirelessly communicate and exchange information with the external controller via any known wireless communication protocol (e.g., Bluetooth communication, infrared communication, near field communication (NFC), radio-frequency (RF) communication, etc.). In embodiments described herein, the internal and external controllers are configured to wirelessly communicate and exchange information via the frequency band of the Medical Implant Communication Service (MICS), which includes frequencies between 402 and 405 MHz. The external controller is configured to run power transmission algorithms (described in greater detail herein) based on data received from the internal controller and push power to the transmission belt from the battery, based on such algorithms. The data provided by the internal controller to the external controller includes various operational parameters associated with the implant as well as the internal battery. Accordingly, the external controller is configured to control the appropriate amount of energy to be delivered to the internal controller (e.g., via coplanar energy transfer between the inductive coils) based on the data received from the internal controller.

The present invention further provides a portable and wearable patient device, such as a smart watch, configured to wirelessly communicate with at least the internal controller and receive data therefrom associated with the operational parameters of the implant and internal battery. Such operational parameters may include, for example, the current status of the implant (e.g., operating metrics, energy demand, etc.) and current status of the internal battery (e.g., remaining useful life of battery, battery faults, etc.). In some embodiments, a patient may not wish to wear the external assembly, as it may be cumbersome and uncomfortable to wear over extended periods of time (e.g., during the day). Accordingly, when the external assembly is not in use (e.g., patient takes off the transmission belt), the internal controller may rely on the internal battery for an energy supply. In such instances, rather than relying on the external controller as a means of providing informational data, the smart watch may be used to collect data from the internal controller and further output informational data to the patient or medical professional providing care to the patient. Accordingly, any crucial information related to operational parameters of the implant and/or internal battery can be communicated to the patient or medical professional without the need for the patient to wear, or otherwise carry, the external controller.

For example, the smart watch may be configured to provide alerts to a patient so as to warn the patient (or medical professional) of any critical information related to the implant or internal battery. For example, based on data received from the internal controller, the smart watch may provide at least one of a visual, audible, and haptic alert indicating a potential failure in the implant or battery. For example, data received from the internal controller may relate to the internal battery having had a fault or potentially having a fault in the future, as well as the amount of useful battery life remaining, thereby indicating a recharge is necessary. The alert may include information about the condition of the battery, such as that it has exceeded a threshold of probability to stop working or to explode. Such warnings can serve to alert the patient to secure some backup power source, or it can alert the patient that surgery is required to replace the defective battery.

Accordingly, this invention provides new approaches for medical implant wireless power transfer and monitoring, which will increase the safety and efficiency, and in parallel reduce the cumbersomeness of traditional TET use by simplify the surgery and placement process.

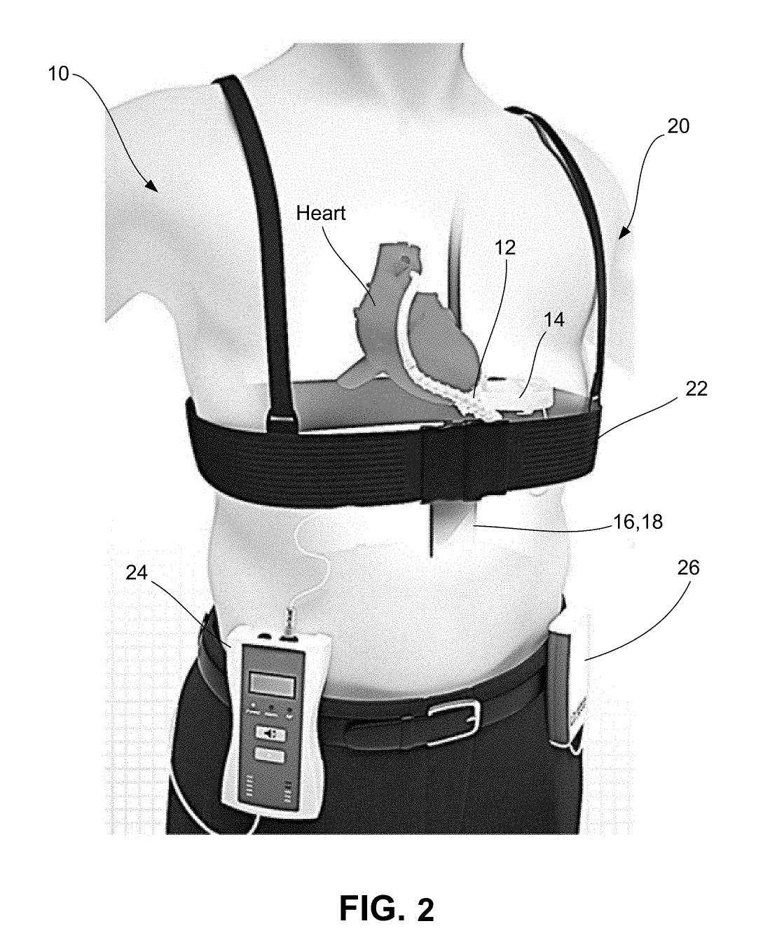

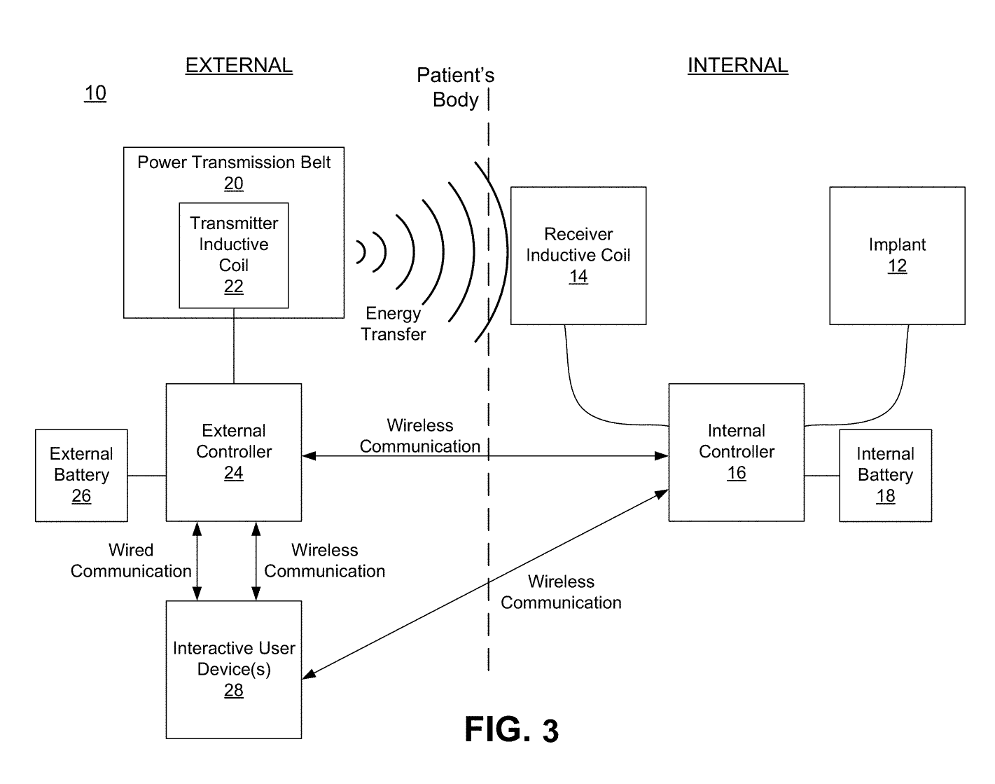

As discussed above, FIG. 1B shows the parts of a wireless coplanar energy system. FIG. 2 illustrates a wireless coplanar energy transfer (CET) system 10 in use on a patient's body. FIG. 3 is a block diagram illustrating the wireless CET system 10. As shown, the system 10 includes an internal assembly, including a medical implant (to be implanted within the body of a patient) and an external assembly (to be provided on the exterior of the patient's body) configured to wirelessly deliver energy to the internal assembly and ultimately provide power to the implant. The internal assembly generally includes, for example, a medical implant 12, such as a ventricular assist device (VAD), to be implanted within the body of the patient. The internal assembly further includes a receiver inductive coil 14 coupled to the implant 12 by way of an internal controller 16 configured to control operation of the implant 12 by way of energy transmitted from the receiver inductive coil 14 or provided by an internal battery 18. The external assembly generally includes a transmission belt 20, which includes a transmitter inductive coil 22, an external controller 24 coupled to the transmitter inductive coil 22 and configured to draw energy from an external battery 26 and control transmission of such energy to the transmitter inductive coil 22, which, in turn, is configured to wirelessly transmit power through the patient's body to the internal receiver inductive coil 14 via magnetic coupling.

For example, FIG. 2 depicts the external transmission belt 20 disposed an individual's torso, and is ideally placed for transmitting power to an implant 12 in the pericardium sack. The transmission belt 20 is designed to be placed externally around a part of the patient's body such that the transmitter inductive coil 22 is disposed in a coplanar manner with the receiver inductive coil 14 to allow for wireless energy transfer from the transmitter inductive coil 22 to the receiver inductive coil 14. During operation of the CET system 10, the external controller 24 is configured to draw energy from the external battery 26 and control transmission of such energy to the transmitter inductive coil 22, which, in turn, is configured to wirelessly transmit power through the patient's body to the internal receiver coil 14 via the magnetic coupling. Upon receiving energy, the receiver inductive coil 14 is configured to transmit such energy to the internal controller 16, which, in turn, is configured to drive, or control operation of, the implant 12, as well as provide energy to the internal battery 18 (e.g., charge the internal battery).

This physical arrangement of the external surrounding transmitter coil 22 and the internally implanted receiver coil 14 (that is disposed at least partially within an imaginary plane cutting through the patient's body and that is formed or defined by the surrounding external transmitter coil) can be referred to as a coplanar arrangement. And the system of the external transmitter coil and the implanted receiver coil thus can be referred to as a CET system.

CET is different than a known and common technique referred to as transcutaneous energy transfer (TET). TET only transfers energy through an area of the skin of a patient to a shallowly-implanted receiver just under that area of the skin. CET, in sharp contrast, involves surrounding the implanted receiver coil by placing or wrapping a transmitter coil completely around the part of the patient's body within which the receiver coil is implanted. If the receiver coil is disposed within the brain of the patient, for example, then CET involves disposing the transmitter coil externally around the corresponding part of the head of the patient such that an imaginary plane defined by the surrounding transmitter coil extends through at least a portion of the brain-implanted receiver coil. If the receiver coil is instead implanted within the descending aorta of the patient's vasculature, CET involves disposing the transmitter coil externally around the corresponding part of the patient's chest such that the imaginary plane defined by the surrounding transmitter coil extends through at least a portion of the aorta-implanted receiver coil. These are just two examples of where the transmitter and receiver coils could be located, and other locations are possible such as the arm or the leg of a patient.

As shown in FIG. 3, the internal and external controllers 16, 24 are configured to wirelessly communicate and exchange information with one another. In particular, the internal controller 16 is coupled to the implant 12 and configured to monitor one or more parameters related to the implant and/or internal battery 18. Such parameters may include, for example, the current status of the implant and/or battery, as well as operational parameters of the implant (e.g., operating metrics, energy demand, etc.) and/or battery (e.g., remaining useful life of battery, battery faults, etc.). In addition to providing output to a patient or medical professional based on receipt of such data, the external controller 24 may be configured to adjust the power supplied to the implant based on the data. For example, the external controller 24 may be configured to run preprogrammed power transmission algorithms (described in greater detail herein) to push power to the transmitter inductive coil 22 from the external battery 26 for the eventual delivery to the implant 12. However, in some embodiments, the external controller 24 may further be configured to adjust power delivery on-the-fly based on the data received from the internal controller 16. Accordingly, in some embodiments, the external controller is configured to control the appropriate amount of energy to be delivered to the internal controller (e.g., via coplanar energy transfer between the inductive coils) based on the data received from the internal controller 16.

The internal and external controllers 16, 24 may also be configured to communicate with one or more interactive user device(s) 28. For example, the at least one of the internal and external controllers 16, 24 may be configured to communicate and share data with a device associated with a patient or medical professional providing care to the patient. The device 28 may be embodied as any type of device for communicating with either of the internal or external controllers and/or other devices over a network. For example, at least one of the user devices 28 may be embodied as, without limitation, a computer, a desktop computer, a personal computer (PC), a tablet computer, a laptop computer, a notebook computer, a mobile computing device, a smart phone, a cellular telephone, a handset, a messaging device, a work station, a distributed computing system, a multiprocessor system, a processor-based system, and/or any other computing device configured to store and access data, and/or to execute software and related applications consistent with the present disclosure.

As shown in FIG. 1B, for example, a tablet computing device 30 may be coupled to the external controller 24 and configured to communicate with the external controller 24 via a wired connection (e.g., USB cable or the like). Additionally, or alternatively, a portable and wearable computing device 32, such as a smart watch, may be configured to wirelessly communicate with at least one of the internal and external controllers 16, 24. It should be noted that devices 30 and 32 can have a wired or wireless connection with the external controller 24, as well as further communicate with one another via any known wired or wireless connection.

The internal and external controllers 16, 24, and interactive user devices 28 may be configured to wirelessly communicate with one another via any known wireless communication protocol. For example, the wireless transmission protocol may include, but is not limited to, Bluetooth communication, infrared communication, near field communication (NFC), radio-frequency (RF) communication, cellular network communication, the most recently published versions of IEEE 802.11 transmission protocol standards as of September 2015, and a combination thereof. In embodiments described herein, the internal and external controllers 16, 24 and either of the tablet and smart watch devices 30, 32 are configured to wirelessly communicate and exchange information via the frequency band of the Medical Implant Communication Service (MICS), which includes frequencies between 402 and 405 MHz.

The tablet computing device 30 may be used by a physician or other medical professional for patient specific configuration of different power transmission modes on the external controller 24 during initialization of the CET system 10 (e.g., coupling of the internal and external controllers with one another and the like). Additionally, or alternatively, the tablet device 30 may be used to receive data from the internal controller 16 by way of the external controller 24 to provide informational data to the patient or medical professional related to the implant or other components of the internal assembly.

The portable and wearable computing device (hereinafter referred to as smart watch 32) is configured to wirelessly communicate with and receive data from the internal controller 16, absent the presence of the external assembly (e.g., transmission belt 20 and transmitter coil 22, external controller 24, and external battery 26). In particular, the smart watch 32 may be configured to wirelessly communicate with at least the internal controller 16 and receive data therefrom associated with the operational parameters of the implant 12 and internal battery 18. Such operational parameters may include, for example, the current status of the implant (e.g., operating metrics, energy demand, etc.) and current status of the internal battery (e.g., remaining useful life of battery, battery faults, etc.). As previously described, in some embodiments, a patient may not wish to wear the transmission belt 20 and external controller 24 and battery 26, as it may be cumbersome and uncomfortable to wear over extended periods of time (e.g., during the day). Accordingly, when the external assembly is not in use (e.g., patient takes off the transmission belt 20 and external controller/battery 24, 26), the internal controller 16 may rely on the internal battery 18 for an energy supply.

In such instances, rather than relying on the external controller as a means of providing informational data to a patient or medical professional, the smart watch may be used to collect data from the internal controller and further output informational data to the patient or medical professional providing care to the patient. Accordingly, any crucial information related to operational parameters of the implant and/or internal battery can be communicated to the patient or medical professional without the need for the patient to wear, or otherwise carry, the external controller, as will be described in greater detail herein.

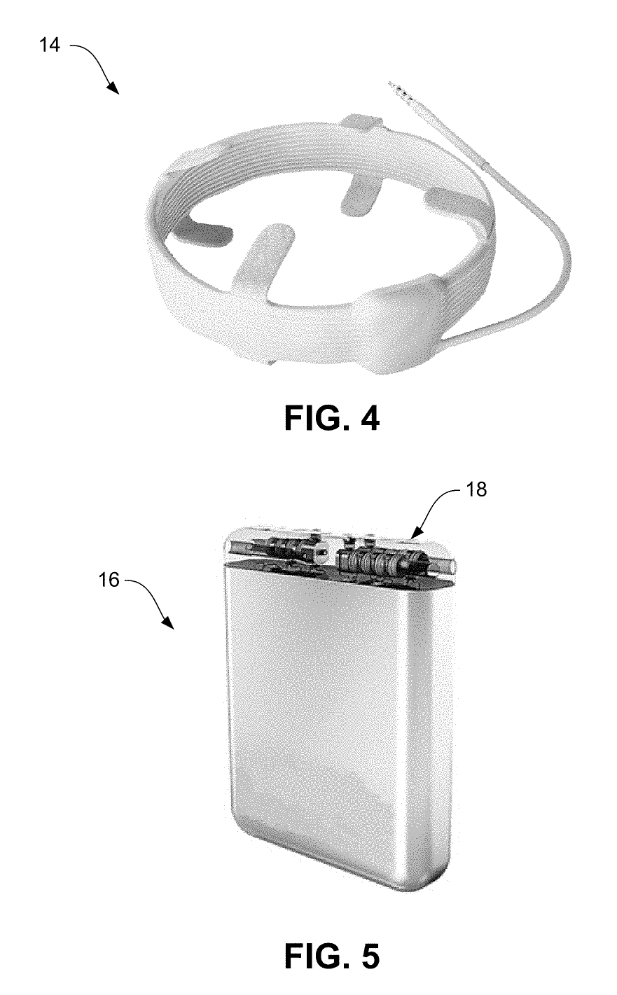

FIG. 4 depicts an embodiment of the receiver inductive coil 14 and FIG. 5 depicts an embodiment of the internal controller 16 including an internal battery 18. It should be noted that specific details regarding each of the implant 12, inductive coil 14, internal controller 16, and internal battery 18 are described in greater detail herein. Generally, the receiver inductive coil 14 is placed around the lung and fixated to the chest wall. The coil 14 possesses state of the art resonance structure technology and high power receiving capability, as described in greater detail herein. Furthermore, in the event that no power is required by the implant 12, the ring static resonance frequency can be changed so as to avoid overpowering. The internal controller is generally configured to control power receiver circuits, activate electronics in the implant 12, such as VAD brushless DC motor, control battery charging circuits, and wirelessly communicate with the external controller 24 and at least the smart watch 32 to provide data related to parameters of the implant and battery. The battery 18 is configured to provide power backup and enables several hours of operation in the absence of the external assembly.

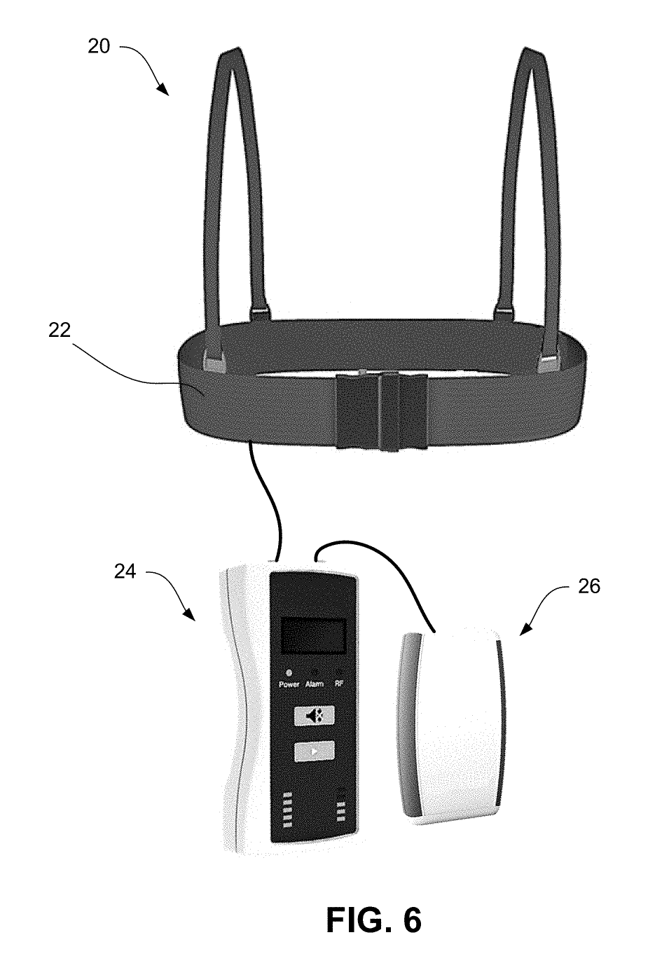

FIG. 6 depicts an embodiment of the external assembly of the system, including the transmission belt 20 (having the transmitter inductive coil 22), the external controller 24, and external battery 26. The belt 20 generally comprise a flexible and durable material configured to be worn over a portion of the patient's body and conform thereto so as to provide adequate contact and positioning of the transmitter inductive coil 22 relative to the implanted receiver inductive coil 14. Furthermore, the transmitter inductive coil 22 may also be composed of an expansive or flexible material so as to allow for stretching to occur to fit over certain portions of the body (e.g., torso, thigh, etc.). Accordingly, a single-size belt 20 may be used and worn by a variety of differently-sized patients. The external controller 24 is configured to run preprogrammed power transmission control algorithms for drawing energy supplied by the external battery 26 and pushing the energy to the transmitter coil 22 using special power driver circuits.

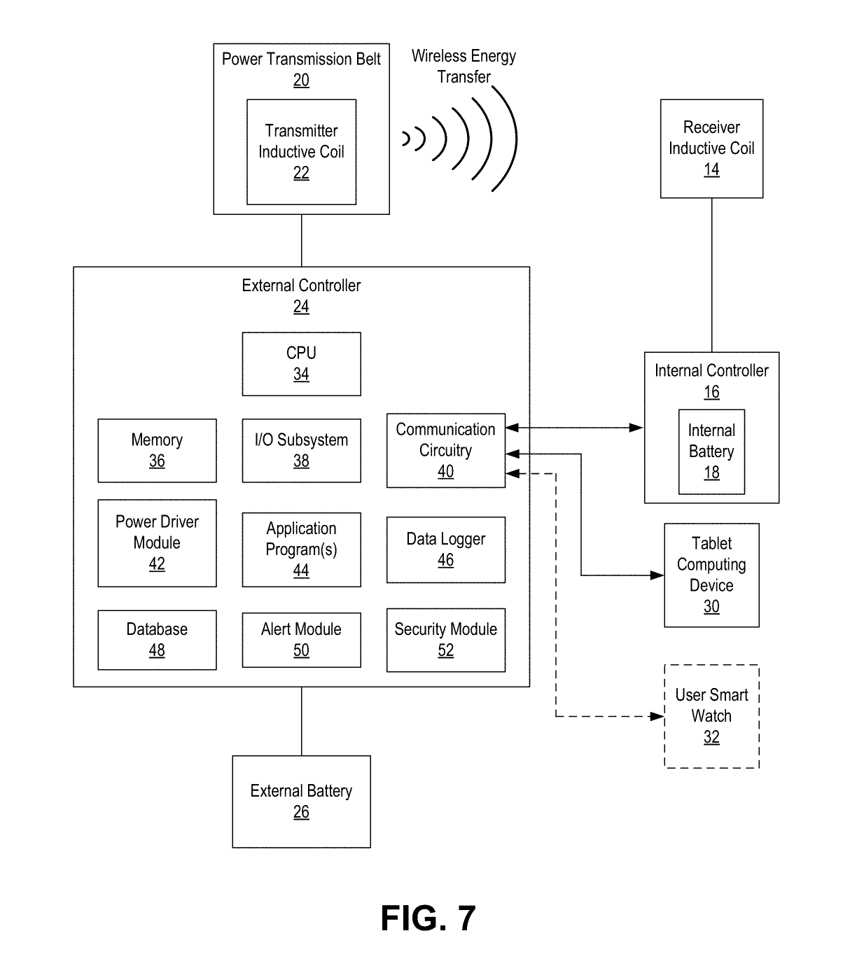

FIG. 7 is a block diagram illustrating the external controller 24 in greater detail. As shown, the external controller 24 includes a computer processing unit (CPU) 34 including one or more processors, a memory 36, an input/output subsystem 38, communication circuitry 40, power driver module 42, one or more application programs 44, a data logger 46, database 48, an alert module 50, and a security module 52. As generally understood, the external controller 24 may include fewer, other, or additional components, such as those commonly found in conventional computing systems. Additionally, in some embodiments, one or more of the illustrative components may be incorporated in, or otherwise from a portion of, another component. For example, the memory 36, or portions thereof, may be incorporated into the CPU 34 in some embodiments.

The CPU 34 may be embodied as any type of processor capable of performing the functions described herein. For example, the processor may be embodied as a single or multi-core processor(s), digital signal processor, microcontroller, or other processor or processing/controlling circuit. Similarly, the memory 36 may be embodied as any type of volatile or non-volatile memory or data storage capable of performing the functions described herein. In operation, the memory 36 may store various data and software used during operation of the external controller 24, such as operating systems, applications, programs, libraries, and drivers. The memory 36 is communicatively coupled to the CPU 34 via the I/O subsystem 38, which may be embodied as circuitry and/or components to facilitate input/output operations with the CPU 34, the memory 36, and other components of the external controller 24. For example, the I/O subsystem 38 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 38 may form a portion of a system-on-a-chip (SoC) and be incorporated, along with the CPU 34, the memory 36, and other components of the controller 24, on a single integrated circuit chip.

The communication circuitry 40 of the external controller 24 may be embodied as any communication circuit, device, or collection thereof, capable of enabling communications between the external controller 24 and at least one of the internal controller 16, the tablet computing device 30, and smart watch 32 via a wired (e.g., for the tablet computing device 30) and wireless transmission protocols (for the internal controller 16 and smart watch 32). The communication circuitry 40 may be configured to use any one or more communication technology and associated protocols, as described above, to effect such communication. For example, the communication circuitry 40 may be configured to communicate and exchange data with at least one of the internal controller 16 and smart watch 32 via a wireless transmission protocol including, but not limited to, Bluetooth communication, infrared communication, near field communication (NFC), radio-frequency identification (RFID) communication, cellular network communication, the most recently published versions of IEEE 802.11 transmission protocol standards as of September 2015, and a combination thereof. As previously described, the internal and external controllers 16, 24 and smart watch devices 30, 32 are configured to wirelessly communicate and exchange information via the frequency band of the Medical Implant Communication Service (MICS), which includes frequencies between 402 and 405 MHz.

The power driver module 42 may be embodied as any type of driver configured to control energy output to the transmitter inductive coil 22 from the external battery 26. The power driver module 42 may generally include DC to AC conversion capabilities, current sensing capabilities, a close power loop, and movement adjustment, as will be described in greater detail herein.

The computing system of the external controller 24 may further include one or more application programs 44 directly stored thereon. The application program(s) 44 may include any number of different software application programs, each configured to execute a specific task. For example, different preprogrammed power transmission modes or schemes with associated algorithms may be stored and selected to push power to the transmitter inductive coil 22 from the external battery 26 for the eventual delivery to the implant 12.

The data logger 46 may generally be configured to collect data received from the internal controller 16 and subsequently stored in the database 48. The database 48 may be embodied as any type of device or devices configured for short-term or long-term storage of data such as, for example, memory devices and circuits, memory cards, hard disk drives, solid-state drives, or other data storage devices. In the illustrated embodiment, the external controller 24 may maintain one or more application programs, databases, media and/or other information in the data storage 48.

The alert module 50 may generally be configured to provide an alert to a patient or medical professional so as to warn of any critical information related to the implant or internal battery. For example, based on data received from the internal controller 16, the alert module 50 may be configured to analyze such data and determine whether such data falls within a predefined range, or otherwise meets a certain threshold, in which critical information must be relayed to the patient or medical professional. For example, with regard to remaining battery life, the alert module 50 may be configured to analyze data sent from the internal controller 16 associated with remaining useful battery life of the internal battery 18. Analyzing of such data may include, for example, a comparison of the received data with a predefined set of data, wherein, if the received data falls below a satisfactory value, the alert module 50 identifies the data as being critical and an alert is then provided. The alert may be in the form of a visual alert (e.g., blinking light), an audible alert (e.g., alarm sound), and/or a haptic alert (e.g., vibration).

The security module 52 may generally be configured to allow pairing of the external controller 24 with either of the internal controller 16 and smart watch 32 based on known security protocols. For example, when attempting to connect the external controller 24 to either of the internal controller 16 or smart watch 32 over a wireless connection, security module 52 may be configured to analyze the devices to determine one or more characteristics of the devices and associated user (i.e. implant identity and patient identity). As generally understood, the security module 52 may include custom, proprietary, known and/or after-developed device recognition and characteristics code (or instruction sets), hardware, and/or firmware that are generally well-defined and operable to receive device data and identify common and unique attributes of a device and/or user. A simple scenario of secure pairing of the external controller 24 with the internal controller 16 and/or smart watch 32 may include matching of a serial numbers or other identifiers between devices (e.g., internal controller or smart watch ID must match external controller ID). By successfully pairing, the external controller 24 is able to communicate and exchange data with either of the internal controller 16 and smart watch 32. Additionally, pairing allows for subsequent transmission of data via advanced encryption services (AES) protocols.

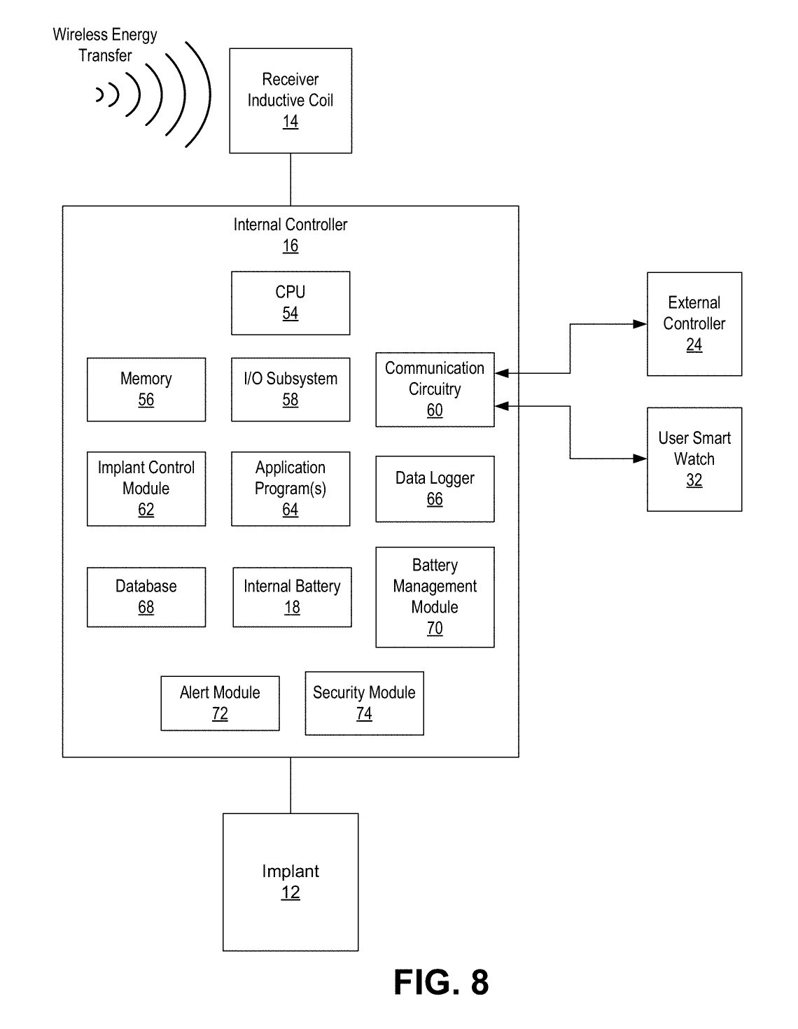

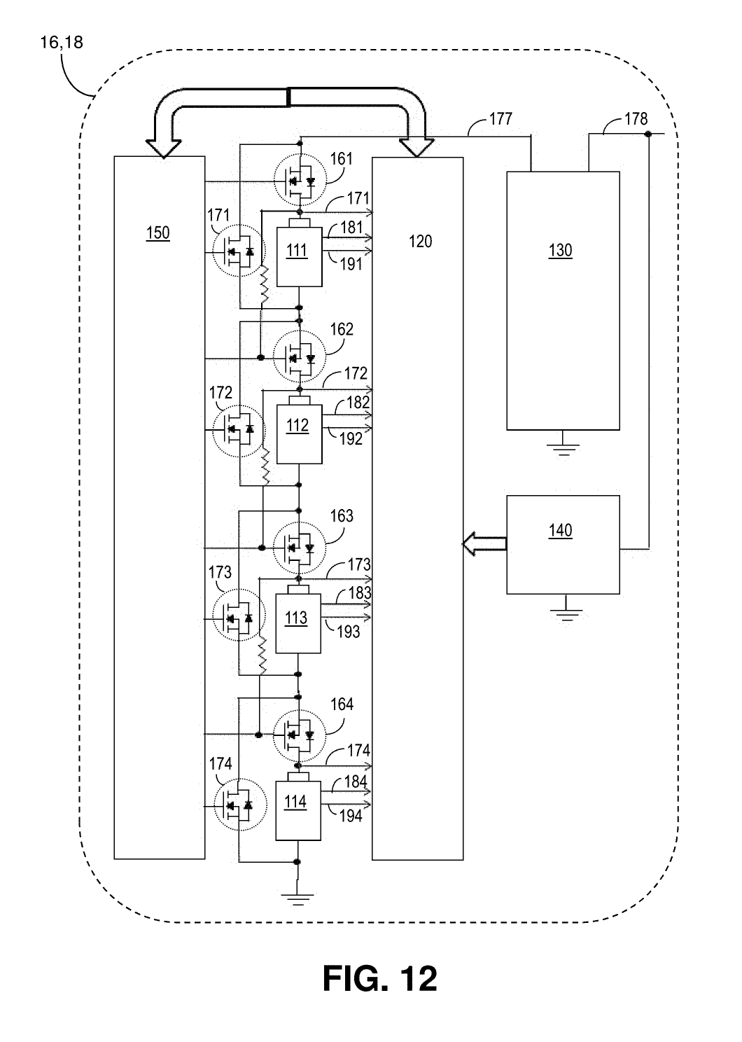

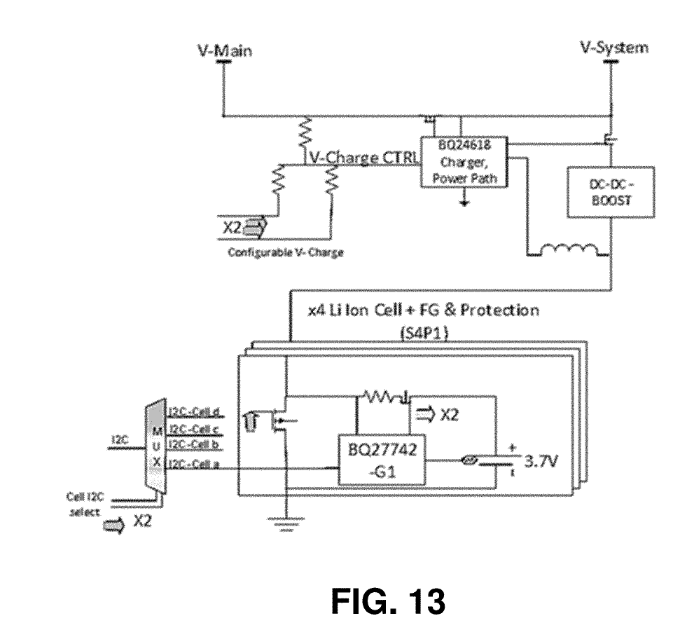

FIG. 8 is a block diagram illustrating the internal controller 16 in greater detail. As shown, the internal controller 16 includes similar computing components as the external controller 24, including a computer processing unit (CPU) 54 including one or more processors, a memory 56, an input/output subsystem 58, communication circuitry 50, one or more application programs 54, a data logger 56, database 58, an alert module 72, and a security module 74. The similar components operate in a similar fashion as previously described. The internal controller 16 includes additional components. For example, the internal controller 16 includes an implant control module 62, and a battery management module 70 (e.g., battery management system). The implant control module 62 is configured to receive energy from the receiver coil 14 and drive, or control operation of, the implant 12 by controlling implant electronics. The battery management module 70 is configured to monitor the status of the internal battery 18 and further control charging and discharge of the internal battery 70, is will be described in greater detail herein with reference to FIGS. 12 and 13.

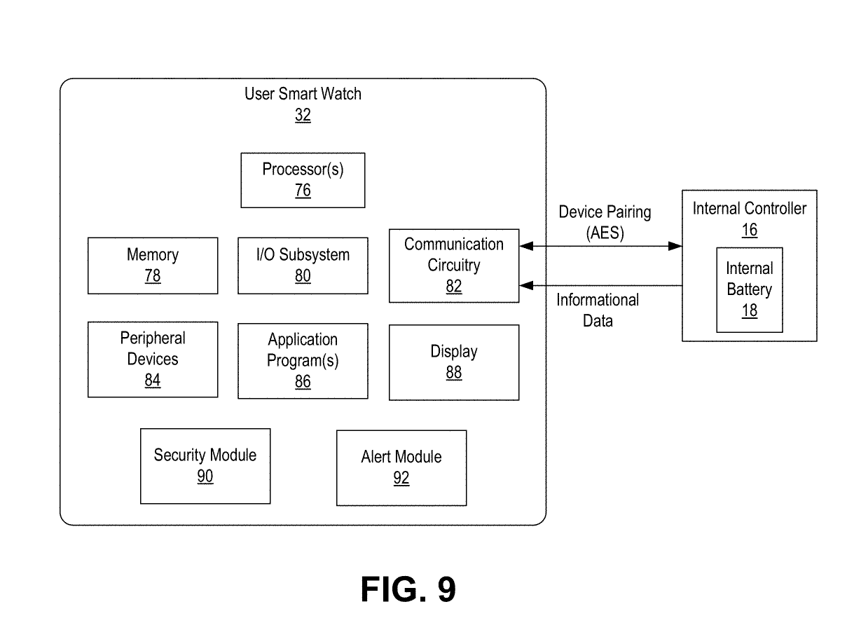

FIG. 9 is a block diagram illustrating the user smart watch 32 in greater detail. As shown, the smart watch 32 includes similar computing components as the external controller 24, including a computer processing unit (CPU) 76 including one or more processors, a memory 78, an input/output subsystem 80, communication circuitry 82, one or more application programs 86, a security module 90, and an alert module 92. The similar components operate in a similar fashion as previously described. The smart watch 32 includes additional components. For example, the smart watch 32 may include one or more peripheral devices 84 and a display 88. The display 88 may generally provide a user interface with which a patient may interact, or otherwise view, informational data associated with the implant 12 and/or internal battery 18. For example, the display 88 may be embodied as a touch-sensitive display (also known as “touch screen” or “touchscreen”) so as to allow a user to interact with a user interface. The peripheral devices 84 may include one or more devices for interacting with the user watch 32 (in addition to the touchscreen display), including a keypad, a microphone, or other input devices. Accordingly, a user may utilize the peripheral devices 84 for interacting with a GUI provided on the display 88 for selection of options of product information.

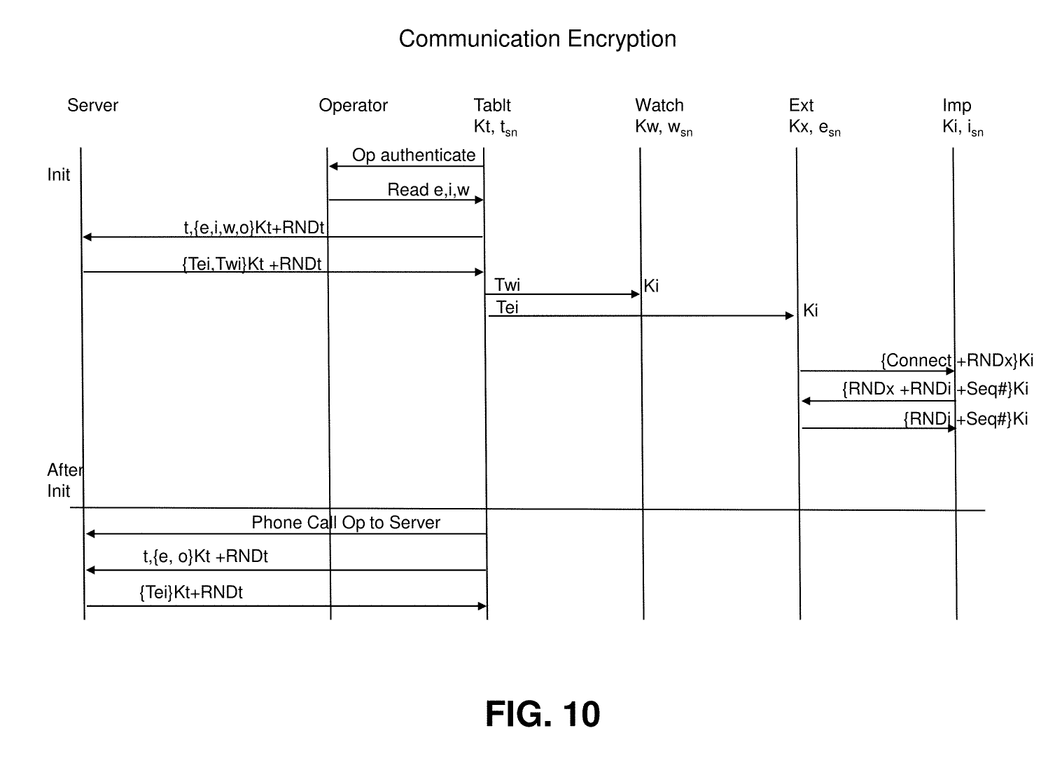

FIG. 10 is a schematic overview of the process of encrypted communication of data between the internal controller 16 and at least one of the external controller 24, the tablet computing device 30, and the user smart watch 32. Upon successfully pairing any one of the devices with another device (e.g., internal controller 16 paired with user smart watch 32), as previously described, a process may ensue for ensuring the encrypted transmission of data from at least the internal controller 16 to the paired device. Such a process is illustrated in FIG. 10.

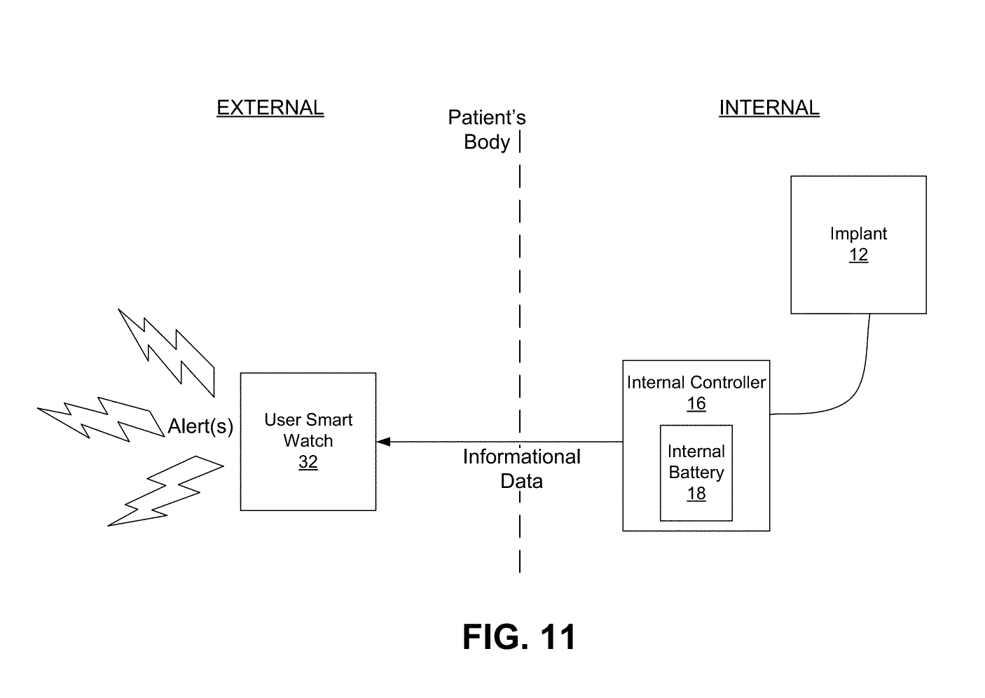

FIG. 11 is a block diagram illustrating the communication of informational data from the internal controller 16 to the user smart watch 32 and the output of alerts to a user based on the informational data. As previously described, a patient may not wish to wear the external assembly, as it may be cumbersome and uncomfortable to wear over extended periods of time (e.g., during the day). Accordingly, when the external assembly is not in use (e.g., patient takes off the transmission belt 20 and external controller 24 and battery 26), the internal controller 16 may rely on the internal battery 18 for an energy supply. In such instances, rather than relying on the external controller 24 as a means of providing informational data, the smart watch 32 may be used to collect data from the internal controller 16 and further output informational data to the patient or medical professional providing care to the patient.

As shown, upon successfully pairing with the internal controller 16, the smart watch 32 is configured to receive data therefrom associated with the operational parameters of the implant and internal battery. Such operational parameters may include, for example, the current status of the implant (e.g., operating metrics, energy demand, etc.) and current status of the internal battery (e.g., remaining useful life of battery, battery faults, etc.). Accordingly, any crucial information related to operational parameters of the implant and/or internal battery can be communicated to the patient or medical professional without the need for the patient to wear, or otherwise carry, the external controller.

Upon receipt of the data, the alert module 92 is configured to analyze the data and determine whether an alert is warranted based on the analysis. For example, the smart watch 32 may be configured to provide alerts to a patient so as to warn the patient (or medical professional) of any critical information related to the implant or internal battery. For example, based on data received from the internal controller 16, the smart watch may provide at least one of a visual, audible, and haptic alert indicating a potential failure in the implant or battery. For example, data received from the internal controller may relate to the internal battery having had a fault or potentially having a fault in the future, as well as the amount of useful battery life remaining, thereby indicating a recharge is necessary. The alert may include information about the condition of the battery, such as that it has exceeded a threshold of probability to stop working or to explode. Such warnings can serve to alert the patient to secure some backup power source, or it can alert the patient that surgery is required to replace the defective battery. Accordingly, this invention provides new approaches for medical implant wireless power transfer and monitoring, which will increase the safety and efficiency, and in parallel reduce the cumbersomeness of traditional TET use by simplify the surgery and placement process.

FIG. 12 shows a diagram of a battery system consistent with one embodiment of the present disclosure. FIG. 13 shows a circuit diagram of a battery system for providing cell redundancy and thermal run away prediction consistent with the present disclosure. The internal controller 16 may generally include a battery management module 70 (such as a battery management system) configured to monitor the status of the internal battery 18 and further monitor operational parameters so as to determine when a recharge is required and whether the battery is functioning properly, thereby identifying potential issues or faults. The structure and properties of the internal battery 18 consistent with the present disclosure are discussed in U.S. Patent Publication No. 2015/0130283, filed Nov. 7, 2014, the content of which is hereby incorporated herein by reference in its entirety.

As shown in FIG. 12, a battery system of the present invention is capable of balancing voltages in response to a fault condition. The system includes four lithium-ion cells 111-114 connected in series. In an embodiment, these can be the 18650 cylindrical-type cells with a nominal voltage of 3.7 V. Other embodiments may include different types of cells, or may include fewer than or more than four cells.

Embodiments of the battery may include various cathodes, anodes, and electrolytes known in the art. For example, the cathode may comprise lithium cobalt oxide (LiCoO₂), lithium nickel manganese cobalt oxide (Li[NiₓMn_{y}Co_{z}]O₂), lithium nickel cobalt aluminum oxide (Li[NiₓMn_{y}Co_{z}]O₂), lithium iron phosphate (LiFePO₄), lithium manganese oxide (LiMn₂O₄), or any other material known in the art. The anode may be graphite or another suitable material. The electrolyte may comprise for example ethylene carbonate, dimethyl carbonate, diethyl carbonate, or a mixture thereof, along with a conducting lithium salt such as LiPF₆, LiBF₄, LiAsF₆, LiCF₃SO₃, or LiClO₄.

The battery-management system (BMS) 120, also known as a controller unit, receives voltage 171-174, temperature information 181-184, and resistance information 191-194 from each cell 111-114. The software of the BMS 120 can be configured to detect when one cell is getting too hot compared to the other cells. It can then respond by isolating the faulty cell from the others, rebalancing the voltages, or taking other steps to mitigate the situation before a thermal runaway or other problematic event can occur. The hardware of the BMS 120 may include thermal sensors, voltage sensors, current sensors, as well as electronic safety circuits that control the charging and discharging of the cells. The BMS 120 measures various cell parameters including current and voltage during operation and the software can determine the state of charge of the cells. In embodiments, the BMS 120 is configured to recognize when a parameter has reached a certain threshold indicative of a pre-fault condition, and respond by taking steps to prolong the operating life of the battery, while simultaneously notifying the user to find another power source.

The transistors can be metal-oxide-semiconductor field-effect transistors (MOSFETs) or any other transistor known in the art. The load switch or driver 150 is on the high side, meaning that it connects the cells to an electrical load, or disconnects them from it. It is coupled to a controller 120, which sends a signal to the high-side driver 150 based on inputs 171-174, 181-184, and 191-194, for example, from cells 111-114. If the controller 120 determines, for example, based on the inputs of cell 111 that there is a fault or there is a potential future fault, the controller signals the high-side driver to electronically isolate or turn off the defective cell 111 by turning off the N-channel MOSFET switch 161.

In one embodiment, the remaining cells 112-114 provide energy to an electronic device (not shown) such as a ventricular assist device (VAD) at the lower voltage that resulted from one cell being turned off. In such embodiments, the VAD would have been designed to accept the lower voltage for operation. Optionally, the system comprises a DC/DC converter or voltage booster 130. If one or more cells are isolated by the BMS 120 due to faults or potential faults, the voltage booster 130 ramps up the voltage of the remaining cells to maintain a normal power level to the VAD or other device. The controller unit 120 performs cell voltage balancing to keep all the cells in a battery pack at close to the same voltage so as to avoid a destabilizing over-charge. In some embodiments this may be accomplished by using switching shunt resistors across the cell to bring high voltage cells into line with the other cells in the pack. The output voltage is maintained at a level required by the boost converter 130, as long as one or more cells are active. This redundant cell design allows the battery to maintain its normal output level in a fault situation. In some embodiments the battery is designed to be able to continue functioning with one or more cells turned off. In other embodiments the battery can continue functioning for only a short time with one or more cells turned off.

In another embodiment of battery system, one of the cells is a reserve cell, which can be connected via a shunt (not shown). The reserve cell can be a backup or spare cell, which is not in use during regular operation of the battery. Alternatively, the reserve cell can have a regular function of powering auxiliary electronics of the VAD or other device. When one of the cells 111-114 fails and has been isolated by the operation described above, the reserve cell is switched on and brought into the series by activating the shunt. In embodiments where the reserve cell's normal function is to provide auxiliary power, the controller 120 assesses the failed or isolated cell to determine whether it is still capable of powering the auxiliary electronics. If it is, the controller 120 proceeds to switch that cell and the reserve cell, so that the reserve cell comes into series with the other active cells to provide power to the device, and the failed cell provides power to the auxiliary electronics. If the failed cell is incapable of powering even the less demanding auxiliary electronics, it remains isolated and the pack of functioning cells is used to power the device and the auxiliary electronics.

In some embodiments the controller 120 can attempt to revive a failed cell by charging it, via slow charge, pulse charge, or another type of charge known in the art. For implantable electronic devices, the type of charge should be compatible with use inside the body. For example, fast charging that results in excessive temperature increase may not be desirable in some embodiments. In embodiments where the cell has not yet failed, but has been determined to be in a pre-failure condition, that pre-failure cell may be revived by the controller 120 in the same manner as described above.

As previously described, the present disclosure provides an alert system for notifying the user when a battery fault has occurred or will potentially occur. Systems of the invention provide differentiable alerts for faults or potential faults of different severity. For example, a small or insignificant fault may trigger a minor alert to keep the user apprised of the battery's condition, whereas a more severe fault may trigger a more emphatic or even painful alert, such as a shock, that underscores the gravity of the fault. Alerts can correspond to potential faults of varying degrees as well.

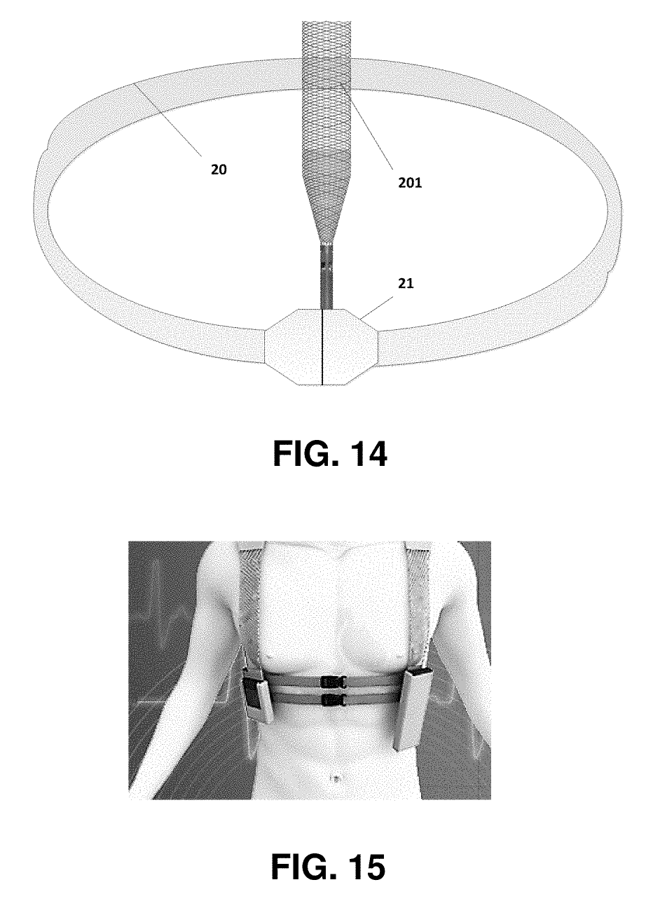

FIG. 14 depicts another view of the coplanar energy transfer system of the invention (also referred to as CET systems). Referring to FIG. 14, a surrounding belt 20 is depicted with a medical stent 201 therein. The stent 201 has built into it or incorporated within it a receiver coil of one or more turns of electrically-conductive material such as copper wire, for example. The belt 20 has in or on it, around its entire length, one or more turns of a transmitter coil. Like the receiver coil, the transmitter coil can have one or more turns of electrically-conductive material such as copper wire, for example. Together, the external belt 20 with the transmitter coil and the implantable medical device (such as a stent) with the receiver coil, can be considered a wireless power transfer system. In use, the transmitter coil can be located externally around the chest of a patient or around some other part of the patient's body such as an arm, a leg, a head, or another part of the patient's torso, and the receiver can be implanted within that part of the patient's body, such that electromagnetic power inductively transmitted from the surrounding coil of the belt 102 reaches and is wirelessly received by the patient-implanted receiver coil from all angles and directions.

The stent 201 of FIG. 14 has built into it or incorporated within it the receiver coil, as indicated previously, and in this regard it is noted that the receiver inductive coil can comprise one or more electrically conductive fibers or strands that are among the various fibers or strands that together constitute the stent 201. These fibers or strands that comprise the receiver inductive coil can be electrical wires and can be coated with an electrical insulator. The receiver inductive coil can be built into or incorporated within the stent 201 in a variety of other ways.

In one embodiment, the receiver coil is not built into the device with which it is associated. In this embodiment, the implantable receiver coil is operatively connected (such as by an electrical wire connection) to the implantable device in order to provide wirelessly-received power to the implanted device. The implantable receiver coil can otherwise be physically separate from and not an integral part of the device itself. In another embodiment, the receiver coil is built into the device with which it is associated. In one embodiment, the receiver coil is a stent 201 as shown in FIG. 14 as the device with which the implantable receiver coil is associated.

In addition to VADs, the receiver coil can be associated with a variety of other types of implantable devices, including, for example, a constant glucose meter (CGM), a blood-pressure sensing device, a pulse sensing device, a pacemaker, implantable cardioverter defibrillators (IDC), digital cameras, capsule endoscopies, implanted slow release drug delivery systems (such as implanted insulin pump) a nerve stimulator, or an implanted ultrasound device.

In operation, the CET system generates lower radio-frequency (RF) energy densities than TET systems. Because CET uses a surrounding external belt-like transmitter coil, the RF energy that is inductively transmitted into the patient's body from the transmitter coil is spread out and not concentrated or focused into or onto a particular spot or area of the patient's body. Using CET, the transmitted energy is spread out over the external transmitter coil of the CET, resulting in transmitted field strength and power density levels that are lower than TET systems. Also using a surrounding external belt-like transmitter coil eliminate misalignment problem and reduce dramatically the misplacement problems.

It is noted that a power source must be associated with the external transmitter coil to provide that coil with the power that it will then wirelessly transmit for receipt by the implanted receiver coil. A controller unit also typically will be provided to regulate the operation of the transmitter coil. Like the transmitter coil, both the power source and the controller will be external to the patient. The external source can be an AC current source, and the transmitter coil can be electrically connected to the AC current source. It also is noted that the transmitter coil can be a transmitter—that is, capable of both transmitting and receiving.

### Providing an Optimal Load to the Receiver Resonance Structure:

In one embodiment according to the invention, the device with which the implantable receiver coil is associated is a ventricular assist device (VAD). In this embodiment a DC-to-DC converter is employed to provide an optimal load to the receiver inductive coil. The DC-to-DC converter is designed to automatically adjust to provide a constant or substantially constant selected optimum load to the receiver inductive coil. Typically, the DC-to-DC converter is implanted within the patient's body along with the receiver inductive coil and the VAD.

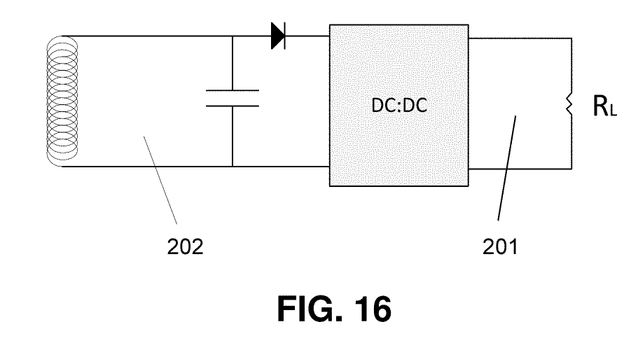

FIG. 16 shows a DC-to-DC converter disposed between the receiver inductive coil and the resonance structure 202 (on the left) and a load R_{L} (on the right). Load 201 may be a VAD or a constant glucose meter, or another implantable device described herein. As shown in FIG. 16, the circuit can also include a half or full-wave rectification (i.e., using a diode or diode bridge). As shown in FIG. 16, resonance structure 202 is formed by the receiver inductive coil and a capacitor. However the external transmitter inductive coil may also be associated with a capacitor to form a transmitter or transmitter resonance structure. A resonance structure of the transmitter can be the same as or different from the receiver.

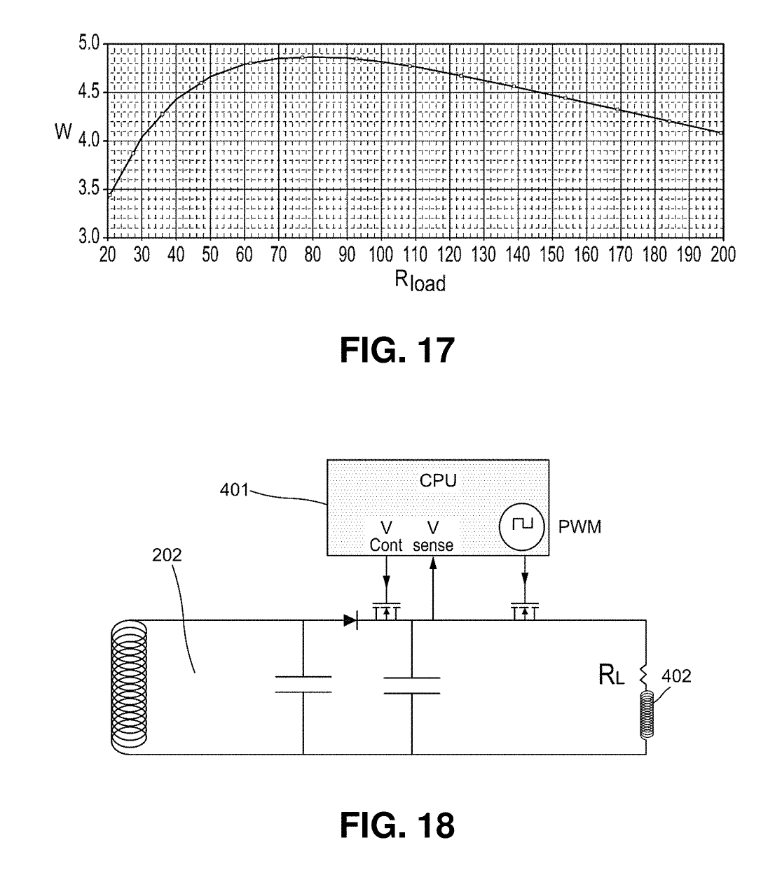

The optimum load can be determined with reference to FIG. 17 which shows a relationship between power harvesting (W) and an R_{load} value for a particular circuit, where R_{load} is the internal resistance of whatever load is associated with the receiver inductive coil. While merely exemplary, the graph of FIG. 17 shows that the best power harvesting for the circuit is 80 Ohms or about 80 Ohms. A load with a resistance lower than 80 Ohms will reduce the voltage on the load and thereby reduce the harvested power, and a load with a resistance higher than 80 Ohms will reduce the current and thereby reduce the harvested power. The shape of the curve in the graph of FIG. 17 is determined by the function (26), provided below. In the case of a VAD, the resistive load represented by the VAD's motor will change as the mechanical load on the motor changes, and the depicted circuit (in FIG. 16) with the DC-to-DC converter is what is used to automatically adjust and provide a substantially constant and optimum load to the receiver inductive coil.

In case of medical implant with high inductive load, like a VAD or implanted slow release drug delivery system that uses a motor, an alternative to the circuit of FIG. 16 is the circuit shown in FIG. 18. The circuit of FIG. 18 can be employed to adjust to an ideal working point of the receiver inductive coil (or, more accurately, of the receiver resonance structure which, as described above, is the combination of the receiver coil and its associated capacitor) when the device with which the implantable receiver coil is associated is a VAD.

An implant with a brushless DC motor, like a VAD, needs adjustable power control to receive exactly the needed mechanical power. As shown in FIG. 17 and by function (26) below and as described above, the best power harvesting is achieved with the optimum R_{load}. In this situation, a high quality motor controller, such as MOTION EN Speed Controller Series SC 1801 F (Faulhaber GmbH & Co. KG, Schonaich, Germany), can be used as a DC-to-DC converter for adjusting the R_{load} to the optimum value using PWM (pulse width modulation). As shown in FIG. 6, a voltage and motor PWM controller 401 gives full control over the working point without any additional measures.

By controlling the voltage and the DC-to-DC rate, the optimum R_{load} can be achieved. The brushless DC motor of the VAD can be simulated with an equivalent resistor and inductor circuit. The speed of the motor is controlled using PWM as the motor input voltage, and the duty cycle is adjusted according to the needed speed. The coils of the VAD's motor flat the current just as is done in DC-to-DC voltage conversion. In this way, the VAD's motor is used as a DC-to-DC converter, and the reflected motor load is dependent on the conversion rate.

Adding a voltage sensor with voltage control adds the capability to select the voltage in the receiver circuit. This gives full control on the reflected load (using the PWM mechanism) and on the used power by controlling the voltage (using the voltage control). For example, a LPC1102 chip can be used for (NXP Semiconductors N.V., Eindhoven, Netherlands) voltage sensing while an internal PWM engine and can control the voltage by using a transistor like SI8409DB (NXP Semiconductors) for closing the inline from the resonance structure 202.

The voltage control can be done in several ways. One example is harvesting control on/off measured, as shown in FIG. 18, in the implanted receiver electronics itself. Another example is transmitting power control in the external transmitter/transmitter primary electronics that closes the loop according to the Vₛₑₙₛₑ in the receiver.

Locking the Receiver and the Transmitter:

Once placed within the body of a patient, the receiver coil shape can be distorted or modified from its at-rest shape and also can move over time to some extent as the patient moves, all depending on the particular location internally within the patient's body where the receiver coil is placed. With changing of its shape, the resonance frequency of the receiver coil changes. It is important for the transmitter resonance structure to be able to automatically find the receiver's resonance and adjust the transmitter's resonance to that found for the receiver and lock to that found resonance. In other words, the transmitter must have the capability to detect the receiver's resonance frequency and then lock to that detected receiver's resonance frequency.

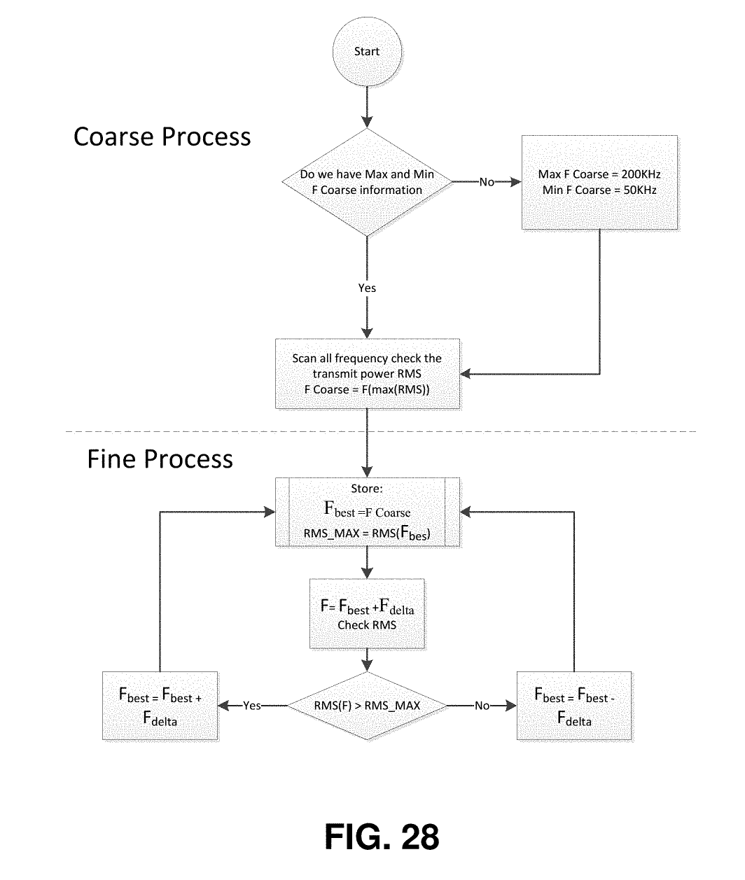

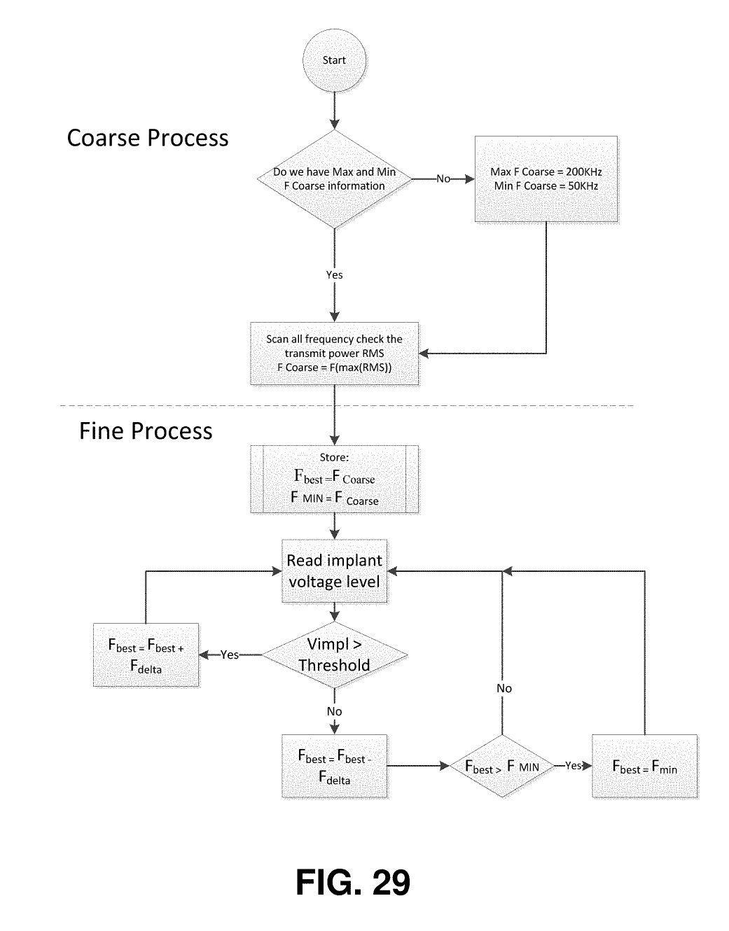

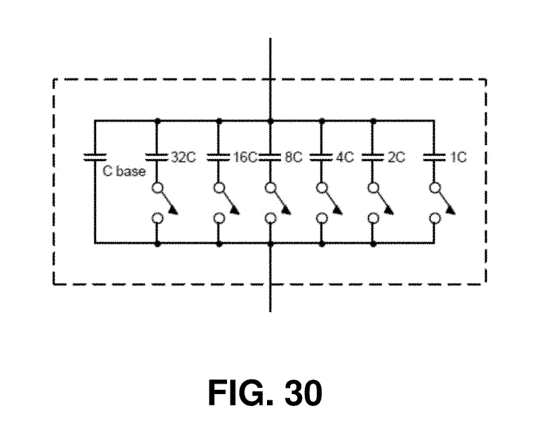

As described in FIG. 28 and in FIG. 29 the transmitter can detect the receiver's resonance frequency in two phases. First, in a coarse phase, when no pre-detected frequency is available, the transmitter uses a fast frequency detection process to roughly detect the receiver resonance frequency or else just start at some predetermined frequency. Second, in a fine phase that occurs after the coarse phase, the transmitter uses an ongoing process of fine tuning to detect the receiver resonance frequency.

The main difference between the two procedures is the simplicity of the solution. FIG. 29 describes a very simple system where the coarse phase detects roughly the resonance, which then becomes the minimum frequency limit. (The system doesn't use the resonance frequency exactly, it uses a frequency above (or below) the resonance and then controls the transfer power by tuning the frequency). This is a simple system and it can work in strong coupling environment like the CET system. In other instances, when the coupling is lower due to distance or receiver/transmitter size/quality it is necessary to use the exact resonance frequency to be able to transfer the needed power.

FIG. 28 describes the fine process that occurs after the first coarse adjust approximately determines the transmitter resonance. In the coarse phase, a microcontroller (MCU) associated with the transmitter resonance structure can have preliminary information about the receiver resonance frequency. The MCU will change the transmitter's driver frequency one after the other and detect the root mean square (RMS) current in the one or more coils of the transmitter. At the end of this phase, the MCU has the result of the entire frequency spectrum, and it can automatically select (as a result of its software programming) the best first coarse frequency, F_{coarse}.

After the coarse phase, the fine phase begins, in which the MCU's software programming dictates the selected frequency from the coarse phase as the best known resonance, F_{best}. Once in the fine phase, the MCU stores the RMS current, adds single F_{delta} to the previous frequency and stores that RMS current. By comparing these two RMS currents, the transmitter's MCU determines whether to add F_{delta} or to reduce F_{delta} from the previous F_{best}. The equation used is as follows: F_{best}=F_{best}+/−F_{delta}. Then, the transmitters' resonance frequency is locked to the receiver's resonance frequency until the next fine phase process occurs. The fine phase process can occur periodically every T_{fine}.

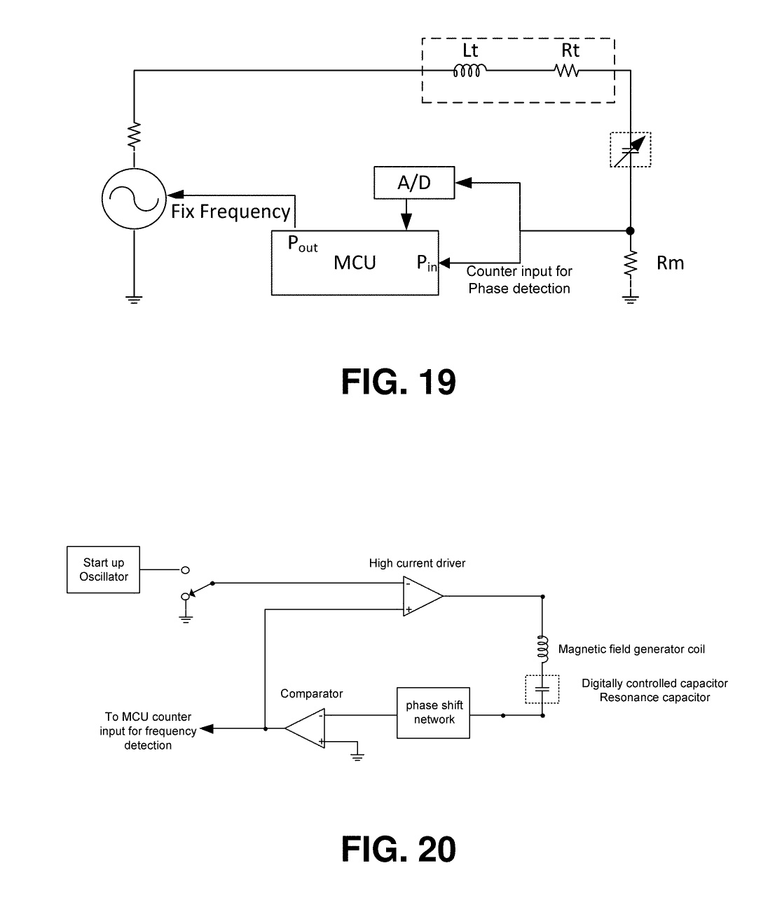

Locking the transmitter resonance to the detected receiver resonance involves the transmitter coil automatically adjusting its capacitors, which can be accomplished using either the circuit shown in FIG. 19, or the circuit shown in FIG. 20, each of which is a resonance LC (inductance and capacitance) structure.

In the circuit of FIG. 19, the MCU forces a fix frequency by generating Pₒᵤₜ pulse in the requested frequency and push the driver circuit. In so doing, the MCU thereby calibrates the configurable capacitor to get resonance. The MCU receives the feedback phase, and adjusts it to the resonance. In resonance, the feedback phase Pᵢₙ should be exact as the generated one Pₒᵤₜ. The MCU then compares the output Pₒᵤₜ to the input Pᵢₙ to validate the resonance. The MCU should adjust the capacitors according to the phase until Pᵢₙ=Pₒᵤₜ

In the circuit of FIG. 20, the circuit is a self-oscillating circuit, and thus is always in resonance; however the MCU can adjust the frequency by changing the capacitors. The MCU can add capacitors to the capacitors array or remove capacitors as described in FIG. 28.

Although FIGS. 19 and 20 show two particular circuits that can be used, it is noted that a variety of variants of phase locked loop (PLL) algorithms and implementing circuits can be used to compensate for impedance changes of the coils by adjusting capacitor value.

### Optimizing Frequency of the Power Transfer:

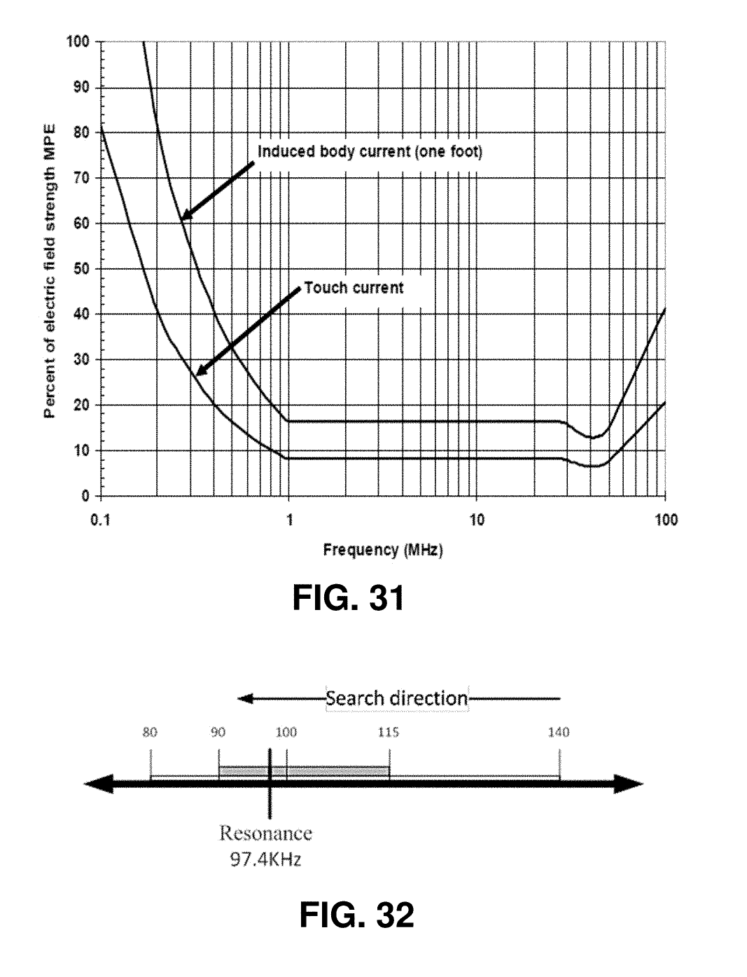

Wireless energy transfer to an implanted medical device, e.g. CET, requires consideration of two parameters, namely (1) the effect of wireless energy transmission on the living tissue through which it is transmitted and (2) the loss in efficiency of the wireless energy transfer due absorption by the living tissue. Other limitations of energy transfer are energy ranges deemed injurious to tissue, as in the ranges set forth in the IEEE Standard C95.1. For example, ranges 3 kHz to 5 MHz can cause painful electro stimulation; 100 kHz to 300 GHz can cause tissue heating (as shown in FIG. 19); and 100 kHz to 5 MHz can cause both electro stimulation and tissue heating. With those parameters and limitations taken into consideration, the optimal frequency of power transmission between a transmitter coil and a receiver coil of the invention provide for high power efficiency and high power transmission without any associated undesirable tissue heating. FIG. 31 shows a percent of electric field strength MPE in the induced body current (of one foot) and the touch current at certain frequencies.

CET systems of the invention achieve optimal energy transfer (such as electromagnetic power) by employing frequencies ranging from 60 KHz to 1 MHz. In that range, the preferred frequency may range from 80 KHz to 300 KHz. In other embodiments, the preferred frequency may range from 90 KHz to 115 KHz. Using frequencies within that range, systems of the invention provide for high power efficiency and high power transmission without any associated undesirable tissue heating.

CET systems of the invention may be programmed to search for a target frequency in order to optimize the functional conditions. This search may be automatic. For the search, the system may utilize circuitry—e.g. a microcontroller (e.g., as in FIGS. 19 and 20), a phase-locked loop circuit (as with analog circuits), or other processor—associated with the transmitter to search and detect the certain frequency of the receiver coil and lock the frequency of the transmitter coil to the receiver coil. The search circuitry controls the transmitter frequency according to input. Depending on the application, the certain frequency may be the resonance frequency or a non-resonance frequency.

The search circuitry may utilize several loop input measurements in order to detect the certain frequency. In addition, the search circuitry may rely on the same principles discussed above to detect the frequency of the receiver and lock the frequency of the transmitter to the receiver. The loop input measurements include, for example, the transmitter power, the receiver power, or other parameters such as an implant current parameter, an implant voltage parameter, an implant charging parameter, a hit parameter, etc. When utilizing transmitter power as an input measurement, the system detects the mutual resonance frequency of the transmitter and receiver at the point where the current in the transmitter inductive coil is maximized. In embodiments that utilize the transmitter power, the circuitry required for the search can be self-contained on the transmitter, and no communication is required from the receiver. When utilizing receiver power as an input measurement, the system detects the mutual resonance frequency of transmitter and receiver at the point where the power within the receiver is maximized, which can be sensed from the current with or without voltage in the receiver circuit. In embodiments that utilize the receiver power or receiver parameters for the search, a microcontroller or other circuitry should also be associated with the receiver to obtain measurements and allow for communication from the receiver to the transmitter.

For resonance frequency searching, the search is initiated by the transmitter circuit. The search utilizes a loop to locate the best frequency with one of the input measurements based on the transmitted efficiency, which is indicated by the current in the transmitter. The search goes down (or up) a range of frequencies (such as the dynamic band), and the resonance frequency is detected because it provides peak efficiency. That is frequencies before and after the resonance frequency are not as efficient, thereby allowing the system to detect the resonance frequency.

For non-resonance frequency searching, the search is initiated by the transmitter circuit. The search utilizes a loop to locate a target frequency with one of the input measurements based on the transmitted efficiency, which is indicated by the current in the transmitter. The search goes down (or up) a range of frequencies, and will stop when it reaches the desired efficiency. By not operating on the resonance frequency, the system is able to control power by manipulating the frequency. For instance, in some CET systems, the desired frequency is 98 KHz just above ideal the resonance frequency of 97.4 KHz. This allows power transfer control simply by changing the frequency. In those instances, the system transmits high power when using a frequency near the resonance and low power when using frequencies farther from resonance.

The resonance and non-resonance frequency searches may be conducted across a dynamic band of frequencies. The dynamic band is the general search range of frequencies. The search typically ends at lowest frequency in the dynamic band. The dynamic band may range from 80 KHz to 140 KHz. The lowest frequency in the dynamic range may be a frequency where no resonance is found. If a system reaches the lowest frequency within the band without finding a resonance frequency of the receiver, the system may terminate the search and may trigger an alarm sound. For example, if 80 KHz is the lowest frequency in the dynamic band, the system will search within the frequency range from the highest frequency to the lowest frequency. In certain embodiments, the search will terminate and an error alarm will sound if the system search reaches the lowest frequency of 80 KHz without finding the resonance frequency of the receiver.

Typically, resonance and non-resonance frequency searches will start at a higher frequency of a dynamic band (e.g., about 140 KHz), and then adjust the search downward towards the resonance or target frequency until the resonance or target frequency is detected. For non-resonance searching, the target frequency may be within range of frequencies called the target main frequencies, which are frequencies desired and targeted by the CET system for optimal efficiency. In certain embodiments, the target main frequency range is from 90 KHz to 115 KHz. It is understood that other frequencies can be used as for the dynamic band and target main frequencies depending on the application (e.g. depending on the selected frequency range).

The following describes using the dynamic band to search for a resonance or target frequency in accordance with FIG. 32. Circuitry associated with the transmitter conducts a search of a dynamic band of frequencies ranging from 140 KHz to 80 KHz. The dynamic band of frequency overlaps with the range of target main frequencies (ranging from 90 KHz to 115 KHz), with the resonance frequency being 97.4 KHz. The transmitter will start search at the top of dynamic band at 140 KHz and then adjust to lower frequencies until the resonance frequency is found or a target frequency within the range of target main frequencies is reached. When conducting a resonance search, the system will initiate an alarm if the search does not find the resonance frequency when it reaches the lower limit of about 80 KHz.

### Placement in a Patient's Body of the Receiver Resonance Structure:

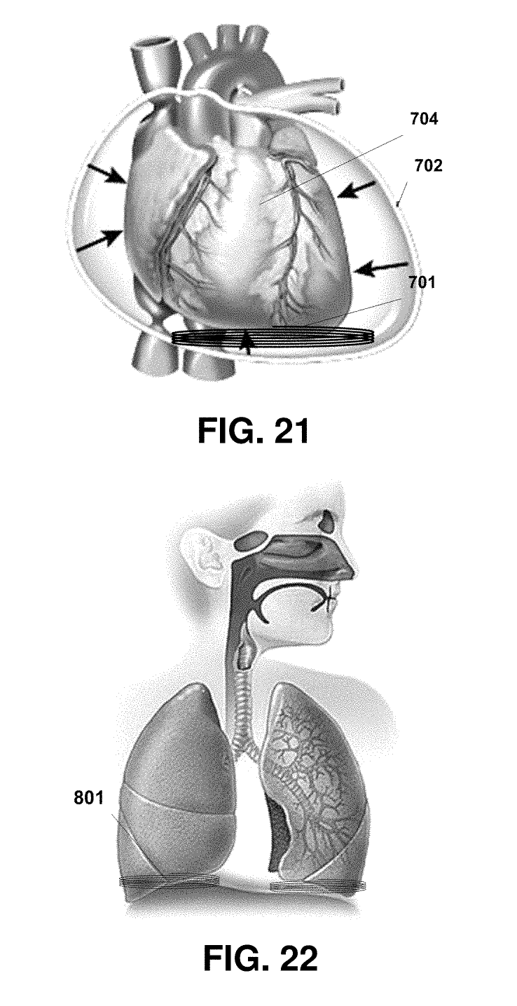

The receiver inductive coil can be placed within the body of a patient at a variety of internal locations. FIGS. 21-23 illustrate three particular examples of a placement location inside the body of a patient.

As shown in FIG. 21, the receiver coil 701 may be placed in the base of the flat part of the pericardia 702, which surrounds the heart 704. The main added value in placing the receiver coil 701 in the pericardia 702 with a VAD is that the pericardia 702 is relatively flat and open in typical VAD surgery. The receiver coil 701 can be glued to the pericardia 702 boundaries, e.g., with surgical glue.

In FIG. 22, it is shown that the receiver coil 801 of a VAD can be placed in the pulmonary cage. One advantage of placing the coil 801 in the pulmonary cage is that the VAD will not disturb the magnetic power harvesting, and that pulmonary cage is relatively easy to access during the VAD surgery.

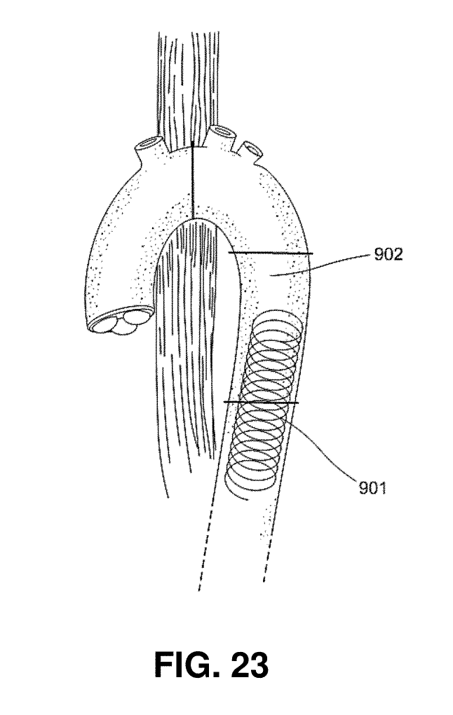

As shown in FIG. 23, the receiver coil 901 may also be placed in an artery 902. The Aorta or the Vena Cava are particularly well-suited for placement of the receiver coil 901 because each is oriented vertically with respect to a plane that cuts in a cross section through the torso of the patient. Placement of the receiver coil 901 in the Aorta or the Vena Cava also allows the receiver coil 901 to be associated with an implantable stent.

### Providing an Optimum Load to the Receiver Resonance Structure:

Having presented various details of various embodiments according to the invention, some theory, equations, and calculations relevant to providing an optimum load to the receiver resonance structure will now be presented.

The ratio between the distance D from the transmitting coil to receiving coil and the wavelength λ is as follows:

$$\frac{D}{λ} = \frac{Df}{c} , \qquad ( 1 )$$

where f is the transmitting frequency and c=3.10⁸ m/s is the speed of light.

Given that the maximum distance Dₘₐₓ does not exceed 0.4 m and the working frequency is f=100 kHz, the ratio Dₘₐₓ/λ=0.00013<<1. Thus, we can conclude that the receiving coil is in the quasi-static area, and we can neglect the effects of the phase difference due to the wave propagation.

The amplitude of the voltage induced in the receiving coil according to the Faraday's law [1] is as follows:

$${v}_{r} ( t ) = - \frac{d Φ}{dt} = - \frac{d}{dt} ( B \cdot a ) , \qquad ( 2 )$$

where Φ is the magnetic flux through the receiving coil, B is the magnetic flux density, and a is the effective area of the receiving coil.

To estimate the maximum induced voltage (2), assume that the receiving coil is located coaxially with the transmitting coil at its center, where the magnetic flux density B can be calculated as follows [1]:

$$B = \frac{{μ}_{r} {μ}_{0} {I}_{t} {N}_{t}}{2 {R}_{t}} sin ( 2 π f t ) , \qquad ( 3 )$$

where μᵣ is the relative permeability of media, μ₀=4π10⁷ V·s/(A·m) is the permeability of vacuum, Iₜ is the amplitude of the current in the transmitting coil, and Rₜ and Nₜ are the radius and number of turns of the transmitting coil correspondingly.

The effective area of the receiving coil can be calculated as follows:

a=πRᵣ²Nᵣ, (4)

where Rᵣ and Nᵣ are the radius and the number of turns of the receiving coil correspondingly.

Substituting (3) and (4) into (2) and differentiating with respect to the time, gives the following expression for the amplitude of the voltage induced in the receiving coil:

$${V}_{r} = 2 π f \frac{{μ}_{r} {μ}_{0} {I}_{t} {N}_{t}}{2 {R}_{t}} π {R}_{r}^{2} {N}_{r} . \qquad ( 5 )$$

The transmitting and the receiving coils can be seen as two coupled inductors, as follows:

$$\{ \begin{matrix}{v}_{t} = {L}_{t} \frac{{di}_{t}}{dt} - M \frac{{di}_{r}}{dt} \\ {v}_{r} = - M \frac{{di}_{t}}{dt} + {L}_{r} \frac{{di}_{r}}{dt}\end{matrix} , \qquad ( 6 )$$

where vₜ and vᵣ are the transmitter and receiver coils voltages, iₜ and iᵣ their currents, and M is the mutual inductance.

Assuming that the current in both coils is a sine-wave of frequency ω=2πf, (6) can be written as follows:

$$\{ \begin{matrix}{v}_{t} = j ω {L}_{t} {i}_{t} - j ω {Mi}_{r} \\ {v}_{r} = - j ω {Mi}_{t} + j ω {L}_{r} {i}_{r}\end{matrix} . \qquad ( 7 )$$

The mutual inductance M can be found from the open circuit experiment, where iᵣ=0:

$$\{ \begin{matrix}{v}_{t} {\vert}_{{i}_{r} = 0} = j ω {L}_{t} {i}_{t} \\ {v}_{r} {\vert}_{{i}_{r} = 0} = - j ω {Mi}_{t}\end{matrix} . \qquad ( 8 )$$

Rearranging the second equation of (8) with respect to M and substituting (2)-(5) gives us:

$$M {\vert}_{{i}_{r} = 0} = - \frac{{v}_{r}}{j ω {i}_{t}} = - \frac{- \frac{d}{dt} ( B \cdot a )}{j ω {i}_{t}} = \frac{{μ}_{r} {μ}_{0} {N}_{t}}{2 {R}_{t}} π {R}_{r}^{2} {N}_{r} = 3.9 µH . \qquad ( 9 )$$

The value of M obtained in (9) increases as a function of the relative permeability μᵣ of the receiver core.

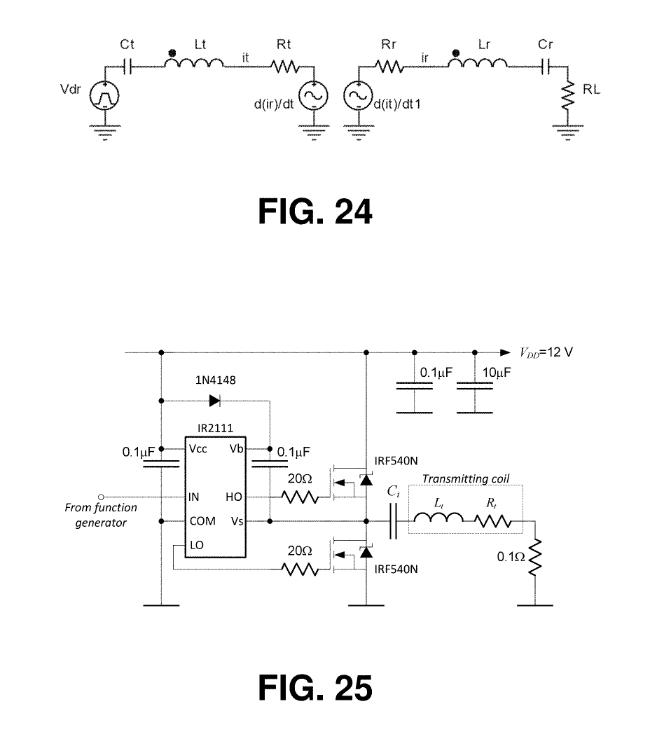

For the purpose of efficiency calculation, assume that the transmitter coil is loaded with a series resonant capacitor and the receiver coil is loaded to form a series resonant circuit as describe in FIG. 24.

The transmitter current is calculated using the coupled-inductor model (7), as follows:

$${i}_{t} = \frac{{v}_{s}}{{R}_{t} + \frac{{( ω M )}^{2}}{{R}_{r} + {R}_{L}}} , \qquad ( 24 )$$

where vₛ=2V_{DD}/π is the effective voltage of the source V_{dr} at the first harmonic of the excitation frequency, Rₜ is the active resistance of the transmitter coil, Rᵣ the active resistance of the receiver coil, and R_{L} is the load resistance.

The amplitude of the load voltage is given by:

$${V}_{L} = \frac{2 ω {MV}_{DD} / π}{{R}_{t} + \frac{{( ω M )}^{2}}{{R}_{r} + {R}_{L}}} \cdot \frac{{R}_{L}}{{R}_{r} + {R}_{L}} = \frac{2 ω {MV}_{DD} / π}{{R}_{t} ( {R}_{r} + {R}_{L} ) + {( ω M )}^{2}} \cdot {R}_{L} , \qquad ( 25 )$$

where V_{DD} is the supply voltage of the half-bridge driver of the transmitter.

From here, the load power is given by:

$${P}_{L} = \frac{{V}_{L}^{2}}{2 {R}_{L}} = \frac{2 {( ω {MV}_{DD} / π )}^{2} {R}_{L}}{{( {R}_{t} ( {R}_{r} + {R}_{L} ) + {( ω M )}^{2} )}^{2}} . \qquad ( 26 )$$

Differentiating (26) with respect to R_{L} gives the load resistance that maximizes the load power, as follows:

$${R}_{Lopt} = {R}_{r} + \frac{{( ω M )}^{2}}{{R}_{t}} . \qquad ( 27 )$$

Substituting (27) into (26) yields:

$${P}_{Lopt} = 0.5 \frac{{( {V}_{DD} / π )}^{2} {( ω M )}^{2} / {R}_{t}}{{R}_{t} {R}_{r} + {( ω M )}^{2}} . \qquad ( 28 )$$

Rearranging (28) with respect to the driver voltage gives:

$${V}_{DD} = \frac{π}{ω M} \sqrt{2 {P}_{Lopt} {R}_{t} ( {R}_{t} {R}_{r} + {( ω M )}^{2} )} . \qquad ( 29 )$$

The input power is:

$${P}_{t} = \frac{{V}_{DD}}{2 π} {\int }_{0}^{π / ω} {i}_{t} ( t ) dt = 2 {( \frac{{V}_{DD}}{π} )}^{2} \frac{{R}_{r} + {R}_{L}}{{R}_{t} ( {R}_{r} + {R}_{L} ) + {( ω M )}^{2}} , \qquad ( 30 )$$

while its optimal value considering (27) is:

$${P}_{topt} = {( \frac{{V}_{DD}}{π} )}^{2} \frac{2 {R}_{r} + {( ω M )}^{2} / {R}_{t}}{{R}_{t} {R}_{r} + {( ω M )}^{2}} . \qquad ( 31 )$$

Dividing (29) by (31) gives the efficiency of the wireless power transmission corresponding to the optimum load resistance:

$${η}_{opt} = \frac{{P}_{Lopt}}{{P}_{topt}} = 0.5 \frac{1}{1 + \frac{2 {R}_{r} {R}_{t}}{{( ω M )}^{2}}} . \qquad ( 32 )$$

The general expression for the efficiency is:

$$η = \frac{{P}_{L}}{{P}_{t}} = \frac{{( ω M )}^{2}}{{R}_{t} ( {R}_{r} + {R}_{L} ) + {( ω M )}^{2}} \cdot \frac{{R}_{L}}{{R}_{r} + {R}_{L}} . \qquad ( 33 )$$

Differentiating (33) with respect to R_{L} gives the load resistance that maximizes the efficiency:

$${R}_{L η max} = \sqrt{{R}_{r}^{2} + \frac{{( ω M )}^{2} {R}_{r}}{{R}_{t}}} . \qquad ( 34 )$$

The maximum efficiency can be calculated by substituting (34) into (33).

$${η}_{max} = 0.5 \frac{1}{1 + \frac{2 {R}_{r} {R}_{t}}{{( ω M )}^{2}}} . \qquad ( 32 )$$

The maximum efficiency and maximum load power for the parallel-loaded receiver is identical to that of the series one. The optimal load resistance and maximizing the efficiency for the parallel-loaded receiver differ from (27) and (34). However, the derivation is similar. The specific formulae for the load resistance is not developed here and instead we find the optimal resistance using computer simulations tool like PSPICE® (Cadence Design Systems, San Jose, Calif.), a full-featured, native analog and mixed-signal circuit simulation tool.

The circuit shown in FIG. 25 is a transmitter. The source Vdr is built from two BUZ11 N-MOSFETs driven by the IR2111 gate driver. The 0.1 Ohm resistor is used for the transmitter current monitoring. Both the transmitter and receiver capacitors are chosen with low ESR. The load resistance is chosen as R_{L}=0.5 Ohm, and the driver voltage V_{DD}=12 V. Substituting these values and the other setup parameters (Rₜ=1 Ohm, Rᵣ=0.65 Ohm, M=2.056 μH) into (26) gives for P_{L}=3.16 W. The measured voltage amplitude on the load resistance is 1.75 V, which corresponds to P_{L}=3.1 W. The input power drawn from the power supply is Pᵢₙ=V_{DD}/π·I=12/3.14·2.8=10.7 W. The efficiency is η=P_{I}/Pᵢₙ=28%. It is noted that the load resistance is not optimized for the maximum output power.

The circuit shown in FIG. 38 is a parallel-loaded receiver. The source V2 is built from two BUZ11 N-MOSFETs driven by the IR2111 gate driver. The 0.1 Ohm resistor is used for the current monitoring. Both the transmitter and receiver capacitors are chosen with low ESR. Substituting the model parameters into (29) gives V_{DD}=11.5 V for P_{L}=5 W. Computer simulations have shown that the maximum load power of 4.85 W is obtained for R_{L}=80 Ohm. This result closely correlates with laboratory measurements, where an output power of 4.5 W was measured for V_{DD}=12 V. The input power drawn from the power supply is Pᵢₙ=V_{DD}/π·I=12/3.14·4.2=16.05 W. The efficiency is π=P_{I}Pᵢₙ=28%.

Inserting a simple single-phase rectifying circuit before R4, as shown in the circuit in FIG. 15, takes about 0.2 W dissipated on the diode with 2 A peak diode current and 44 V peak diode reverse voltage. The peak voltage on the receiver capacitor is 25 V, and the peak voltage on the transmitter capacitor is 500 V.

### Optimizing the Design of the Resonance Structure of the Transmitter and Receiver

Several design factors contribute to the quality of the transmitter and receiver resonance structures of CET systems of the invention. Particularly, the resonance structure (e.g. resonance structure 202 as shown for the Receiver in FIG. 16) can be designed to minimize loss during the energy transfer. The quality of the resonance structure is dependent on the ratio between the inductance (L) to the resistance (R) of the coil. In radio frequency (RF) couplings, the quality of the resonance LC structure is dependent on the ratio of the inductance and capacitance (LC) to the resistance (R) of the coil. Those ratios are referred to herein as the quality factor (Q). Resonance LC structures are depicted in FIGS. 19 and 20. A higher Q indicates a lower rate of energy loss relative to the stored energy of the resonance structure. The quality factor (Q) of the coil originates in the coil's ohmic resistance, which can be calculated knowing the material and thickness of the coil, the diameter of the coil, D, the number of turns of coil, N, and the magnetic effects—the skin effect and the proximity effect (described below). In addition to the regular resistance and loss factors, a design may also take into consideration the skin effect and the proximity effect and the capacitor internal resistance.

The quality factor Q of the receiver and/or transmitter resonance structures can vary to provide optimum energy transfer. In certain embodiments, the quality factor of the transmitter is within the range of about 100 to about 500, and the quality factor of the receiver is within the range of about 50 to about 200.

In certain embodiments, the quality factor of the coils is improved with nongalvanic connected coils.

The following describes the various parameters that influence the Q of RF couplings.

The first parameter is a capacitor's or capacitors' equivalence series resistance (ESR) of the resonance structure of either the transmitter or receiver coils. For optimal Q, a capacitor's or capacitors' ESR in a resonance structure is less than the coil's active resistance. In certain embodiments, the capacitor's or capacitors' ESR is less than 5 times the coil's active resistance.

Another parameter that influences Q is the skin effect. The skin effect is the tendency of an alternating electric current (AC) to become distributed within a conductor such that the current density is largest near the surface of the conductor, and decreases with greater depths in the conductor. That is, the electric current flows mainly at the “skin” of the conductor, between the outer surface and a level called the skin depth. The skin effect causes the effective resistance of the conductor to increase at higher frequencies where the skin depth is smaller, thus reducing the effective cross-section of the conductor.

In order to reduce the skin effect in transmitter and receiver coils, the coils can be constructed using wires configured to transmit alternating currents, such as litz wire. A litz wire is a type of cable used in electronics to carry alternating current, and are made according to the “litz wire standards.” Litz wires consist of many thin wire strands, individually insulated and twisted or woven together, following one of several known patterns often involving several levels (groups of twisted wires are twisted together, etc.). Litz wires suitable for use in systems of the invention include those manufactured by New England Wire Technologies (Libson, N.H.). Litz wire standards relate the number of internal strands to the wire structure. Litz wire sizes are often expressed in abbreviated format: N/XX, where N equals the number of strands and XX is the gauge of each strand in AWG (American Wire Gauge). Wires suitable for use in the invention have a gauge of 36-48 AWG (with the preferred gauge being 38-40 AWG). In certain embodiments, the external coil is a wire with 100-600 strands with a gauge of 36-48 AWG; and the internal coil is a wire with 100-400 strands with a gauge of 36-48 AWG. In preferred embodiments, the internal coil and the external coil are formed from a wire with 175 strands/40 AWG.

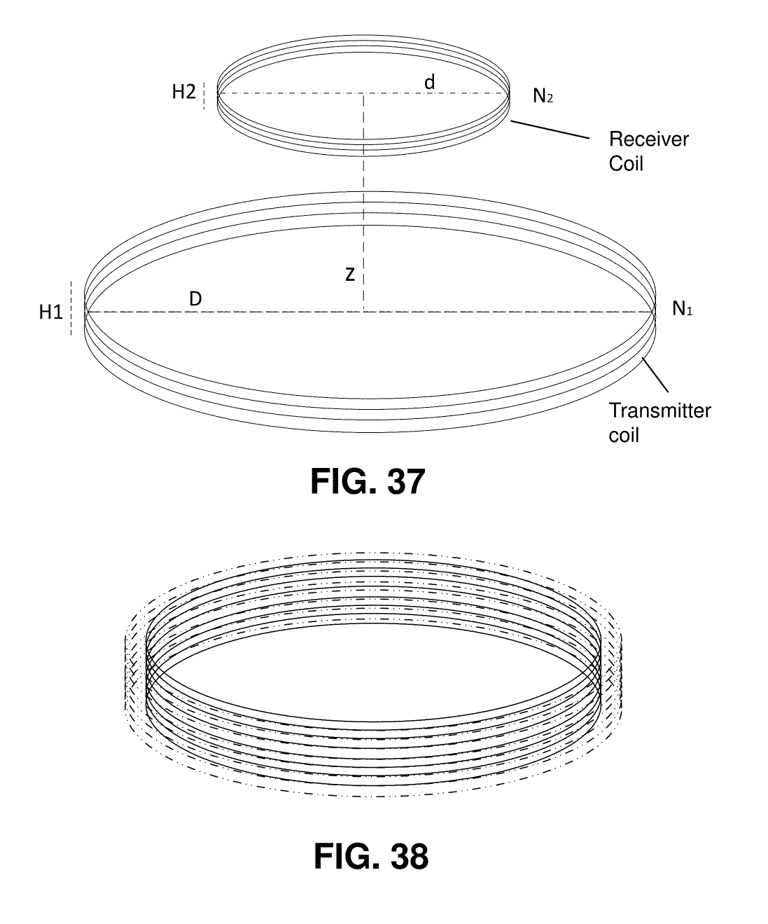

Another parameter that influences Q is the proximity effect. Proximity effect is the tendency for current to flow in loops or concentrated distributions due to the presence of magnetic fields generated by nearby conductors. The proximity effect is most evident in a conductor carrying alternating current, if currents are flowing through one or more other nearby conductors, such as within a closely wound coil of wire, the distribution of current within the first conductor will be constrained (or crowded into) to smaller regions. This current crowding is known as the proximity effect. This crowding gives an increase in the effective resistance of the circuit. The resistance due to the proximity effect increases with frequency. The proximity effect, in transmission and receiver coils relates to H1, H2 and coil center to center distance z (see FIG. 37). In the end, the proximity effect cannot be uncoupled from geometry, and must be calculated for a given design.

The following are additional optimization features that can also be incorporated into the transmitter and receiver of the CET systems of the invention.

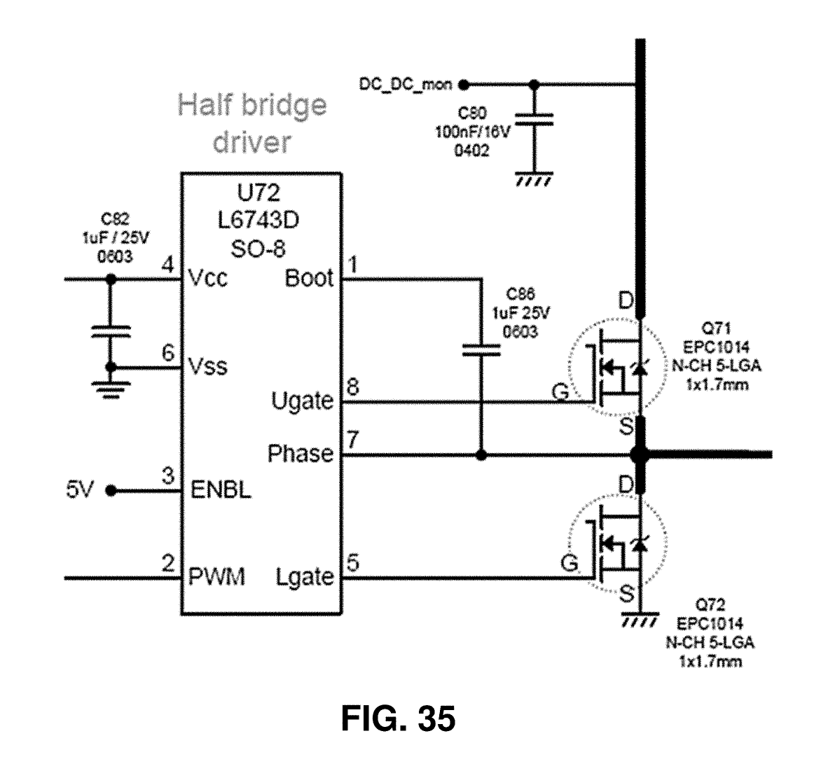

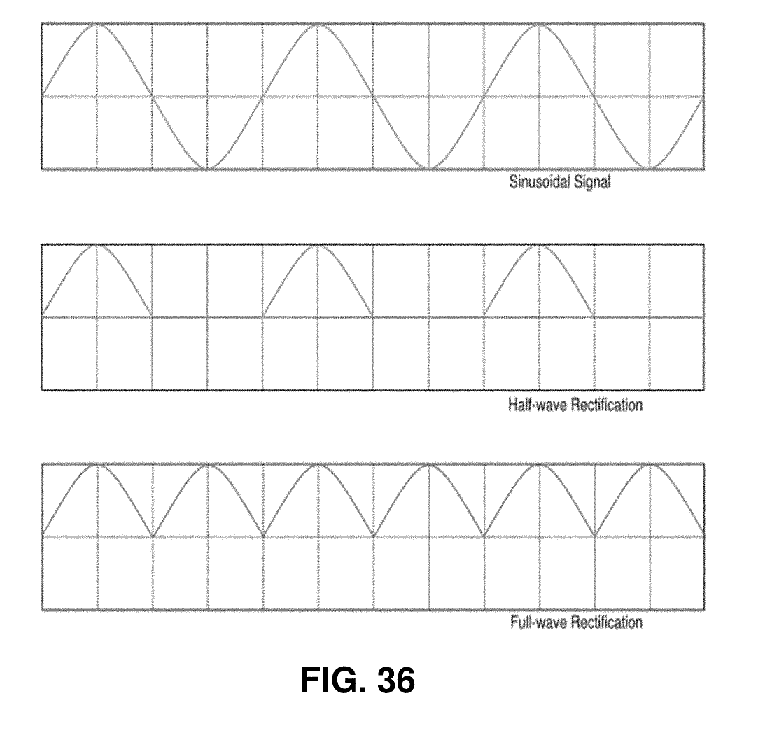

In certain embodiments, the transmitter is optimized by incorporating a square generator, such as a half bridge pulse generator. The half-bridge pulse generator assists with pushing power from the transmitter to the receiver. FIG. 35 depicts a preferred circuit for to half bridge pulse generator. While the circuit depicted in FIG. 35 is a square wave generator, the circuit can generate regular sinusoidal waves in the transmitter coils.

Another optimization feature includes the incorporation of a field-effect transistor in the receiver and/or transmitter. A field-effect transistor (FET) is a transistor that uses an electric field to control the shape and hence the conductivity of a channel of one type of charge carrier in a semiconductor material. Particularly, FETs with low resistance when in saturation can greatly reduce losses due to heating.

In addition, an AC-to-DC conversion circuit may be included in the receiver in order to convert the AC voltage from an AC power source to DC voltage. The AC-to-DC conversion is a process known as rectification. In some embodiments, diodes can be utilized in systems of the invention to reduce loses in the AC to DC conversion circuit. Regular AC to DC conversions involve diode-based rectification circuits. Any rectifier may be used to convert AC voltage to DC current. In certain embodiments, CET systems may utilize a single diode rectification circuit or a diode bridge rectification circuit with the receiver. FIG. 24 illustrates rectification with one diode (half wave) and with a diode bridge (full wave). A diode bridge is an arrangement of four (or more) diodes in a bridge circuit configuration that provides the same polarity of output for either polarity of input. The advantage of the diode bridge is that it uses the entire input wave rather than only half of it.

According to some embodiments, the receiver includes a single diode rectifier. The effect of the single diode will be the same as using a diode bridge because the energy of the closed cycle of the single diode is not lost. Instead, the energy is kept the resonance structure for the active cycle. By using a single diode, only part of the duty cycle is used, and the potential difference in the resonance circuit will be significantly higher than built-in voltage drop across the diodes (around 0.7 V for ordinary silicon p-n junction diodes and 0.3 V for Schottky diodes). Thus, a single diode is able to reduce the transaction. In addition, the ratio of the diode's built-in voltage drop to the total voltage will be better. Also, the efficiency of the diode is related to the diode's size. Therefore, a system utilizing one diode in a rectifier of a certain size is more efficient than four diodes of the same size. In certain embodiments, the diode is a Schottky diode. Schottky diodes minimize the transition cycle loss.

### Effect of a Coplanar Wireless Energy Transfer System's Design and Geometry on Power Transmission

The geometry of the CET systems can affect the efficiency and robustness of the system. Accordingly, the geometry of certain systems can be optimized in accordance with the medical device being powered by a CET system. For example, the type of medical device, its power needs, and the implantation site are inputs that can be used to shape the geometry of a system for optimal energy transfer.

The following are key parameters that effect geometry and design of a CET system of the invention for use with an implant, such as a ventricular assist device (VAD). These systems include generally transmission of power from a belt or a vest transmitter that circumscribes the body in the same plane of the receiver. Optimal key parameters are suggested for and based on a typical VAD having a power requirement of 5 W-20 W and a peak of 30 W. In addition, the optimal key parameters for transmitters and receivers for use with a typical VAD are described in further detail separately below.

FIG. 37 depicts a layout of a CET system for use with a VAD. This layout is helpful for understanding geometry dependent factors. The distance Z, as shown in FIG. 37, is the distance between the centers of the transmitter and receiver coils. In general, the smaller the distance Z is, the better the coupling between the transmitter and the receiver. Preferably, the distance Z is 7 cm or less. Ideally, the receiver coils are concentric with the transmitter coils such that the distance Z is minimized. However, systems described herein maintain power transfer efficiency while allowing distance Z of about 7 cm between the centers of the transmitter and the receiver.

In addition, the diameters of transmitter and receiver coils (see FIG. 37) effect power transmission in a system. Diameter D is the diameter of the external transmitter, which depends and can be adjusted based on the body size of a patient. Diameter d is diameter of the internal receiver. The diameter d of the internal transmitter can be varied depending on the type and placement of the device. As diameter d increases, so does the quality of the coupling between the transmitter and receiver. The ratio of the receiver diameter d to the transmitter diameter D also affects wireless power transmission. In general, a higher diameter ratio increases the quality of the coupling for energy transfer.

Further, the number of wire turns of the transmitter coil N1 and receiver coil N2 also influences power transmission. The greater number of turns of wire in either coil improves magnetic/electronic conversion. However, the resistance caused by the number of wire turns should also be taken into consideration.

Other design considerations that influence power transmission are the capacitor's ESR and the type of wire used (both of which were discussed in further detail above). For optimization, the capacitor's ESR should be as low as practical, and a litz wire should be used to minimize skin effect.

A. Transmitter Geometry and Design

The following are preferable design and geometry details of the transmitter.

According to certain embodiments, the diameter of the transmitter coil D, as shown in FIG. 37, can be sized to fit around an individual's body. In certain embodiments, the diameter D ranges from, for example, about 20 cm (for children) to about 60 cm (overweight adult).

In certain embodiments, the transmit frequency may be in the range of about 60 KHz to about 1 MHz. As discussed in more detail above, the CET system can be designed to search across a range of frequencies such that the transmitter couples to the resonance frequency of the receiver. This search may be automatic. For automatic frequency searches, the range of frequencies searched (e.g. a dynamic band of frequencies) are narrower than the range of the transmit frequency. In addition, a transmitter may be set at a target frequency or a resonance frequency. The dynamic band may be between 80 KHz and 300 KHz. In other embodiments, the dynamic band may be 80 KHz to 140 KHz. The target frequency may be a frequency within the range of 90 KHz to 115 KHz.

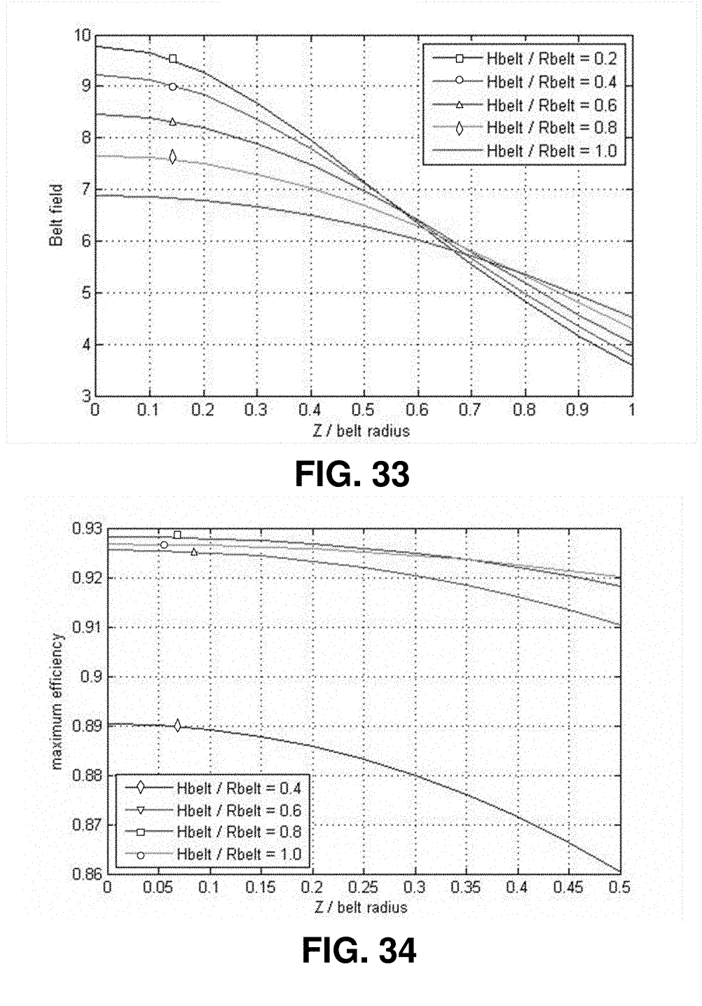

The height H1 of the transmitter coil can be designed to minimize proximity effect. Ideal heights H1 for minimizing proximity effects range from about 3 cm to 20 cm. FIG. 33 illustrates the effect that the height of a transmitter coil has on robustness of power transfer if the proximity effects are ignored. FIG. 34 illustrates the effect that the height of a transmitter coil has when the proximity effects are considered.

In certain embodiments, the height H1 is 0.4-1.5 times the size of the radius (the radius being half of the diameter D). That is, a ratio between the height H1 of the transmitter coil and the radius (D½) of the transmitter coil is 0.4-1.5. For example, if transmitter coil has a radius of 15 cm, the ideal height H1 ranges from 6-22.5 cm. In some embodiments, the ratio between the height H1 and the radius (D½) is about 0.6, 0.8, or 1. Preferably, the ratio is in the range of about 0.8-1. In certain embodiments, the height H1 for optimum efficiency is 12 cm.

According to certain embodiments, the transmitter diameter D is substantially larger than the distance Z between the center of the transmitter and receiver coils. This configuration reduces the effect of dynamic changes in distance Z during coplanar wireless energy transfer (e.g. due to movement of the transmitter compare to the receiver within the body). As a result, power transfer is more reliable and continuous despite the change in Z. The combination of a) transmitter diameter D larger than the distance Z with b) a height H1 to radius (D/2) ratio in the range of 0.8-1 further improves energy transfer.

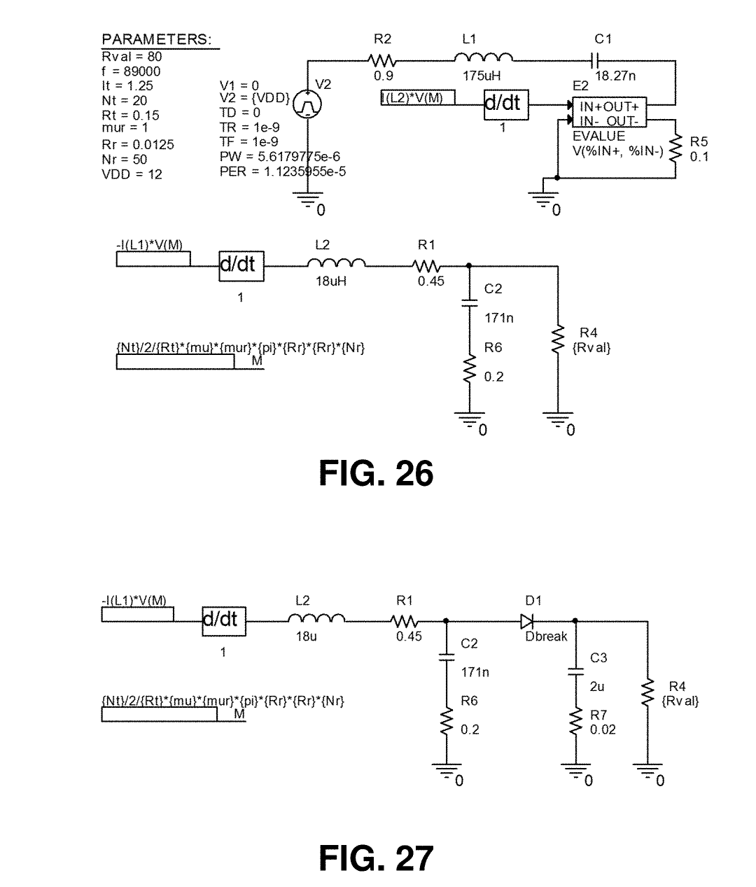

The influence of the number of turns N1 on power transmission depends on the type of wire used and the quality factor of the coil. Using litz wires with 100-600 strands/36-48 AWG, the number of turns N1 of the transmitter coil may range from 6 to 35, preferably 20. In addition, the number of turns N1 may be arranged in 1-3 layers. FIG. 26 depicts a transmitter coil having 16 wire turns arranged in two layers (e.g. 8 turns per layer).

In certain embodiments, a capacitor of the transmitter should have an ESR that is less 5× the coil's active resistance and preferably 1/10 of the Coils resistance. Preferred wires for the transmitter are litz wires with 100-600 strands/36-48 AWG.

B. Receiver Geometry and Design

The following are preferable design and geometry details of the receiver.

According to certain embodiments, the diameter d of the receiver coil, as shown in FIG. 37, can be sized to fit to an anatomic location within the patient. For a receiver coil located in the pericardium, the diameter d may range from, for example, about 7 cm (children pericardium) to about 20 cm (adult pericardium and one pleural cavity).

The ideal number of turns N2 of the receiver coil may range from 5 turns to 50 turns. The turns may be arranged in one to three layers (similar to the transmitter coil depicted in FIG. 38). Preferably, the receiver coil includes 20 turns in one layer.

Like the transmitter coil, the receiver coil can be designed to overcome the proximity effect. There are two different receiver coil designs that can be used to overcome the proximity effect. These designs can also be applied to the transmitter coil. The first design is a connected ring coil and the second design is a separated ring coil. Both designs may include a covering around the inductive wires. The covering is preferably biocompatible and provides insulation. Suitable materials include polymers, such as silicone (e.g. NiSil Med 4735 or Med 421), or epoxy materials. Ideally, the receiver coil is able to collect power for the implant, while maintaining enough flexibility to rest within the diaphragm area.

A connected ring coil design is one in which the coil's wire loops or turns are united such that the distance (i.e. pitch) between each turn is substantially constant. In certain embodiments, a single covering layer connects the coil's turns into a single connected structure. Connected ring coils have some flexibility, but the basic structure of the ring coil is maintained with enough rigidity to ensure that the distance (i.e. pitch) between each turn is substantially constant. Having a uniform minimum distance is best for reducing proximity effect. Pitch is the distance between the center of one turn and the next. The structure of the connected ring coil avoids/minimizes proximity effect of electromagnetism. The flexibility of the connected ring coil can be altered to suit, for example, its intended implantation area. The flexibility depends on the materials chosen for the wire and covering—varying based on those materials' parameters for elongation, tensile, durometer, hardness. In one embodiment, the structure of the connected ring coil has a height of about 1 cm to about 3 cm and a pitch of about at least 0.1 inch. In other embodiments, the pitch is at least 0.5 inches. In some embodiments, the pitch between turns is chosen to be substantially equal to the diameter of wire, which acts to further minimize proximity effect. Connected ring coils of the invention ideally have a narrow, small range of resonance frequency, making it easier to gauge and adapt to the resonance frequency. This narrow resonance frequency characteristic simplifies the calibration and control of the system.

A separated ring coil design includes turns (i.e. rings) that are separated to allow variable pitch and other movement between the turns. This provides more flexibility to the receiver geometry. In order to allow variable pitch and other movement, the turns of the coil are not fully connected to each other such that the rings can move away from each other to a desired extent. For example, only a portion of the turns is coupled so there is more movement between the rings. In other words, a separated ring coil is a coil that has one or more connecting points between turns (or rings), which act to provide flexibility in one or more directions, while preventing over-expansion and over-compression. The movement can be in the pitch direction (e.g. movement between turns) or can be in the lateral direction (upward or downward movement of side by side turns). The flexibility of the separated ring coil can be altered to suit, for example, its intended implantation area. The flexibility depends on the materials chosen for the wire and covering—varying based on those materials' parameters for elongation, tensile, durometer, hardness.

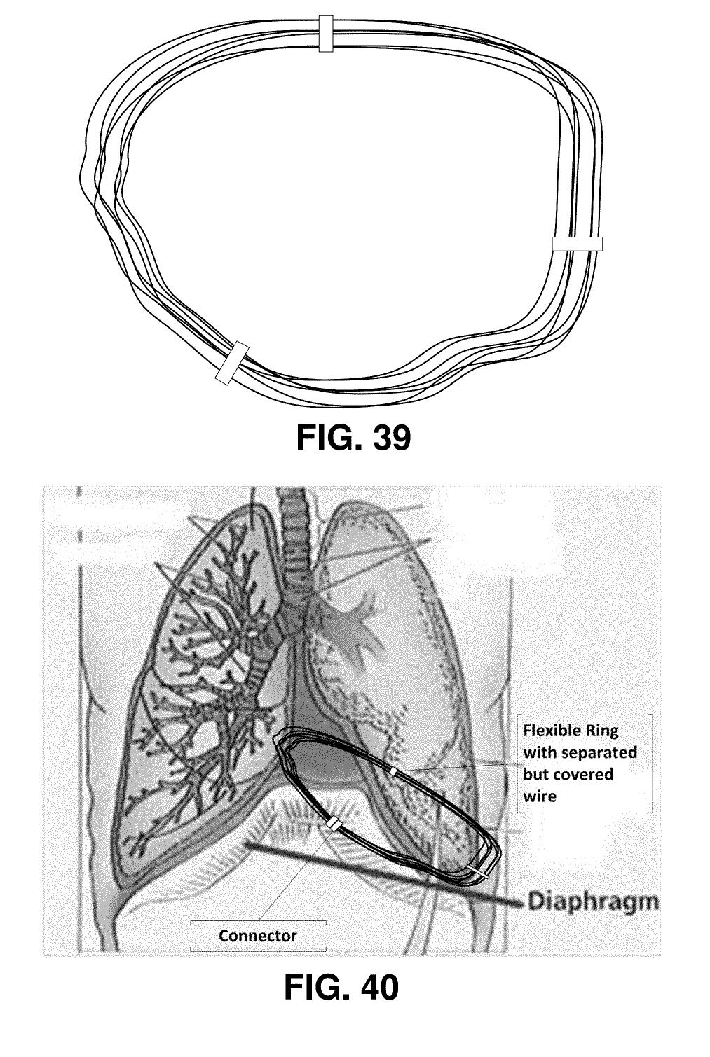

The separated ring coil may also have a covering for insulation, but the covering does not form a single connected structure. Instead, the covering may only partially connect the rings together at one or more points. In certain embodiments, the covering does not connect the rings together, but one or more separate connectors are placed on the coil to connect the rings together (such as the connectors shown in FIGS. 39 and 40). The benefit of the connectors is that a doctor can place them on or manipulate their positions on the ring coil during implantation of the transmitter within the body. This makes it easier to implant the transmitter within the body (such as in the pericardium, pleural cavity, or both). FIG. 40 depicts a separated ring coil disposed within a pleural cavity and pericardium.

While the variable pitch and added movement of separated coils allows for easier implantation, the variable pitch may result in proximity effect if the wires are too close to each other. In order to prevent proximity effect, the invention provides for covering the wire of the separated ring coil with a covering material of a certain thickness to provide a minimal pitch distance between rings. For example, the covering of the ring coil may be 0.05 inch thick, such that the pitch between any two rings touching each other is at least 0.1 inches. The second receiver coil design for reducing pitch is use of separate but covered flexible wires for the receiver coil. In other embodiments, separated but covered wires are used for the receiver coil.

Various modifications may be made to the embodiments disclosed herein. The disclosed embodiments and details should not be construed as limiting but instead as illustrative of some embodiments and of the principles of the invention.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smart phones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed is:

1. A system for monitoring an implantable ventricular assist device (VAD) in a patient, the system comprising:
an implantable assembly comprising a controller and a battery, the controller configured to provide power from the battery to the implantable VAD and collect data associated with at least one of the implantable VAD and the battery; and
a wristwatch comprising: a wireless receiver configured to wirelessly receive the data from the controller; and a data output component configured to present information to a user based on the received data, the information comprising an alert indicating an event that is life-threatening to the patient.

2. A system for monitoring an artificial heart in a patient, the system comprising:
an implantable assembly comprising a controller and a battery, the controller configured to provide power from the battery to the artificial heart and collect data associated with at least one of the artificial heart and the battery; and
a wristwatch comprising: a wireless receiver configured to wirelessly receive the data from the controller; and a data output component configured to present information to a user based on the received data, the information comprising an alert indicating an event that is life-threatening to the patient.

3. The system of claim 1, further comprising:
an external transmission inductive coil and an external controller; and
an internal receiver inductive coil coupled to the VAD and configured to receive wirelessly transmitted energy from the external transmission inductive coil.

4. The system of claim 1, wherein the information is associated with operational or performance characteristics of the implanted medical device or the battery.

5. The system of claim 4, wherein the operational or performance characteristics are selected from the group consisting of operating metrics, energy demand, remaining useful life, fault potential, and a combination of at least two thereof.

6. The system of claim 1, wherein the data output component comprises at least one of a visual display, an audio source, and a haptic feedback source.

7. The system of claim 1, wherein the alert comprises at least one of a visual, audible, and haptic alert.

8. The system of claim 1, wherein the wristwatch comprises a smart watch.

9. The system of claim 1, wherein the controller and the wristwatch are configured to wirelessly transmit data via a wireless transmission protocol selected from the group consisting of Bluetooth communication, infrared communication, near field communication (NFC), radio-frequency identification (RFID) communication, WiFi, and cellular network communication.

10. The system of claim 1, wherein the controller and the wristwatch are configured to wirelessly transmit data via the frequency band of the Medical Implant Communication Service (MICS), or Medical Device Radiocommunications Service (MedRadio).

11. The system of claim 10, wherein the wristwatch serves as a protocol bridge between the implant and off-the-shelf computing device using MICS or MedRadio to communicate with the implant controller and Bluetooth or WiFi to communicate to the off-the-shelf computing device.

12. The system of claim 1, wherein the implantable assembly further comprises a receiver inductive coil coupled to the controller and configured to wirelessly receive inductively-transferred electromagnetic power from a non-implanted power source and provide power to the implanted implantable VAD via the controller.

13. A wristwatch for monitoring operation of an implanted ventricular assist device (VAD), the wristwatch comprising:
a wireless receiver configured to pair with a transmitter implanted within a patient and receive from the transmitter data related to operating parameters of the implanted VAD or an associated implanted battery; and
a data output module configured to alert a user when the operating parameters are indicative of a life-threatening event.

14. The wristwatch of claim 13, wherein the operating parameters comprise operating metrics, energy demand, remaining useful life, fault potential, battery capacity, battery capacitance, battery voltage, battery power, or any combination thereof.

15. The wristwatch of claim 13, wherein the alert comprises at least one of a visual, audible, and haptic alert.

16. The wristwatch of claim 13, wherein the alert comprises an amount of time remaining before failure of the implanted battery.

17. The wristwatch of claim 13, wherein the transmitter is configured to wirelessly transmit data to the non-implanted wireless receiver via a wireless transmission protocol selected from the group consisting of Bluetooth communication, infrared communication, near field communication (NFC), radio-frequency identification (RFID) communication, wifi, and cellular network communication.

18. The wristwatch of claim 13, wherein the transmitter is configured to wirelessly transmit data to the non-implanted wireless receiver via the frequency band of the Medical Implant Communication Service (MICS) or Medical Device Radiocommunications Service (MedRadio).

19. The wristwatch of claim 18, wherein the wristwatch serves as a protocol bridge between the implant and off-the-shelf computing device using MICS or MedRadio to communicate with the implant controller and Bluetooth or WiFi to communicate to the off-the-shelf computing device.

20. A system for monitoring operation of an implanted left ventricular assist device (LVAD) and an implanted battery for conveying blood through a human heart of a patient, the system comprising:
a wristwatch containing a wireless receiver configured to:
pair with a transmitter implanted within the patient, and
receive from at least one implanted processor associated with the transmitter, indications of operating parameters of the LVAD including an indication of an amount of time remaining until reconnection to an external power source is required, and warning signals relating to at least one dangerous state of the LVAD; and
an alarm in the wristwatch for alerting the patient to a life-threatening event during operation of the LVAD.

21. The system of claim 20, further comprising a display on the wristwatch for providing feedback on operation of the LVAD, the feedback including an indicator of an amount of time remaining until the patient is required to reconnect to an external power source.

22. The system of claim 21, wherein the indicator of the amount of time is a display of remaining capacity, capacitance, or voltage of the implanted battery.

23. The system of claim 20, wherein the event comprises a high power event, low implanted battery power, a cessation of operation of the LVAD, or a failure that requires use of redundancy mechanism.

24. The system of claim 23, wherein the event include a disconnection of a connector, a failure of an implanted battery, or a failure of an engine in the LVAD.

25. The system of claim 20, wherein the wristwatch is configured to establish an authenticated secure connection with the implant controller.

26. The system of claim 20, wherein the wristwatch is configured to establish an encrypted connection with the implant controller.

27. The system of claim 20, wherein the wristwatch includes at least one processor for causing to appear on the display instructions to the patient for taking corrective action to mitigate the event.
